SEQUENCE LISTING (6 sequences)

SEQ ID NO: 1 - protein, 31 aa, Homo sapiens
HAEGTFTSDVSSYLEGQAAKEFIAWLVKGRG

SEQ ID NO: 2 - protein, 40 aa, Heloderma suspectum
HGEGTFITSDLSKQMEEEAVRLFIEWLKNGGPSSGAPPPS

SEQ ID NO: 3 - protein, 29 aa, Homo sapiens
HSQGTFTSDYSKYLDSRRAQDFVQWLMNT

SEQ ID NO: 4 - protein, 31 aa, synthetic construct
  REGION 1..31: note = Synthetic
  MOD_RES 2: note = Aib
  MOD_RES 20: note = The amino acid residue is modified with a substituent.
HXEGTFTSDVSSYLEGQAAKEFIAWLVRGRG

SEQ ID NO: 5 - protein, 31 aa, synthetic construct
  REGION 1..31: note = Synthetic
  MOD_RES 2: note = Aib
  MOD_RES 20: note = The amino acid residue is modified with a substituent.
  MOD_RES 31: note = The amino acid residue is modified with a substituent.
HXEGTFTSDVSSYLEGQAAKEFIAWLVRGRK

SEQ ID NO: 6 - protein, 33 aa, synthetic construct
  REGION 1..33: note = Synthetic
  MOD_RES 2: note = Aib
  MOD_RES 21: note = The amino acid residue is modified with a substituent
  MOD_RES 30: note = The amino acid residue is modified with a substituent
HXEGTFTSDVSSYLEEQAARKFIEWLVRGKGEG

(19) United States
(12) Patent Application Publication
(10) Pub. No.: US 2024/0099981 A1
(43) Pub. Date: Mar. 28, 2024

(54) SOLID COMPOSITIONS COMPRISING A GLP-1 AGONIST AND A SALT OF N-(8-(2-HYDROXYBENZOYL)AMINO)CAPRYLIC ACID

(71) Applicant: Novo Nordisk A/S, Bagsvaerd (DK)

(72) Inventors: Betty Lomstein Pedersen, Glostrup (DK); Birgitte Nissen, Ballerup (DK)

(21) Appl. No.: 18/386,839

(22) Filed: Nov. 3, 2023

Related U.S. Application Data
(63) Continuation of application No. 16/570,723, filed on Sep. 13, 2019, now Pat. No. 11,833,248.
Application No. 16/570,723 is a continuation of application No. PCT/EP2019/052487, filed on Feb. 1, 2019.

(30) Foreign Application Priority Data
Feb. 2, 2018 (EP) 18154913.0

(51) Int. Cl. A61K 9/20; A61K 38/26; A61P 3/04; A61P 3/10; C07K 14/605

(52) U.S. Cl. CPC A61K 9/2013; A61K 38/26; A61P 3/04; A61P 3/10; C07K 14/605

(57) ABSTRACT

The invention relates to pharmaceutical compositions comprising a peptide, such as a GLP-1 peptide and a salt of N-(8-(2-hydroxybenzoyl)amino)caprylic acid. The invention further relates to processes for the preparation of such compositions, and their use in medicine.

### CROSS-REFERENCE TO RELATED APPLICATIONS

This application is a continuation of U.S. application Ser. No. 16/570,723, filed Sep. 13, 2019, which is continuation of an International Application No. PCT/EP2019/052487 (WO 2019/149880), filed Feb. 1, 2019, which claims priority to European Patent Application No. 18154913.0, filed Feb. 2, 2018; the contents of which are incorporated herein by reference.

### TECHNICAL FIELD OF THE INVENTION

The present invention relates to solid compositions comprising a GLP-1 agonist and a salt of N-(8-(2-hydroxybenzoyl)amino)caprylic acid, their method of preparation and their use in medicine.

### INCORPORATION-BY-REFERENCE OF THE SEQUENCE LISTING

The instant application contains a Sequence Listing which has been submitted in XML format via the USPTO patent electronic filing system and is hereby incorporated by reference in its entirety. Said XML file, created on Oct. 25, 2023, is named 170097US02.xml and is 11 kilobytes in size.

### BACKGROUND

Human GLP-1 and analogues thereof have a low oral bioavailability. Exposure and bioavailability of human GLP-1 and analogues thereof is very low following oral administration. Human GLP-1 (and analogues thereof) can thus only be detected in plasma after oral administration if formulated with certain absorption enhancers in a specific amount.

Steinert et al. (Am J Clin Nutr, Oct 2010; 92: 810 — 817) discloses oral administration of a tablet comprising GLP-1(7-36)amide and 150 mg sodium N-(8-(2-hydroxybenzoyl)amino)caprylate (SNAG).

WO 2010/020978 discloses an oral pharmaceutical composition comprising a protein and N-(8-[2-hydroxybenzoyl) amino)caprylate (SNAG). Patent applications disclosing oral dosage forms of GLP-1 analogues containing a salt of N-(8-(2-hydroxybenzoyl)-amino)caprylate include WO2012/080471, WO2013/189988, WO2013/139694, WO2013/139695 and WO2014/177683.

Despite these findings there is still room for a further optimized pharmaceutical composition for oral administration of a GLP-1 agonist such as a GLP-1 analogue comprising a substituent.

### SUMMARY

The present invention relates to a composition comprising a GLP-1 agonist and an absorption enhancer or delivery agent. The composition according to the invention in an embodiment includes a very high content of the delivery agent and a minimal content of further excipients as described herein below. The provided compositions display an accelerated absorption, enabling fast and efficient uptake of the active pharmaceutical ingredient.

Oral administration of therapeutic peptides is challenging due to the rapid degradation of such peptides in the gastrointestinal system. Described herein are pharmaceutical compositions providing accelerate absorption of the GLP-1 agonist within 15-30 minutes after administration and thereby improved exposure of the GLP-1 agonist by oral administration. The inventors have surprisingly found that the plasma exposure of GLP-1 agonists increases when compositions are prepared with a very high content of the absorption enhancer and a minimal content of any further excipients.

In an aspect the invention relates to a composition wherein the weight ratio of the delivery agent relative to the total composition, or in particular, relative to the other excipients of the composition, is very high.

In one embodiment, the invention relates to a pharmaceutical composition comprising a GLP-1 agonist, a delivery agent and/or absorption enhancer such as SNAC, wherein the delivery agent/absorption enhancer constitutes at least 90%, such as at least 95% w/w of the excipients of the composition.

In one embodiment, the invention relates to a pharmaceutical composition comprising a GLP-1 agonist, a delivery agent and/or absorption enhancer such as SNAC, wherein the delivery agent/absorption enhancer constitutes at least 90% w/w of the composition.

In additional embodiments, the composition further includes a lubricant.

In an aspect the invention relates to a method of preparing a pharmaceutical composition as described herein such as a method comprising the steps of;

a) granulating a mixture comprising the delivery agent, the GLP-1 agonist and optionally a lubricant and

b) compressing the granulate obtained in step a) into tablets and optionally adding further lubricant to the granulate prior to compression.

In a further aspect the invention relates to a composition or a granule as defined herein for use in medicine, such as for treatment of diabetes or obesity, wherein said composition is administered orally.

In a further aspect the invention relates to a method of treating diabetes or obesity comprising administering the composition as defined herein to a patient in need thereof, wherein said composition is a tablet and is administered orally.

### BRIEF DESCRIPTION OF DRAWINGS

FIG. 1 shows fast dissolution of A1 compared to Reference A.

FIG. 2 shows increased dose-corrected exposure following oral tablet dosing to beagle dogs of analogues A, B and C when formulated according to the invention. Data are given as mean. Compositions A1, B1 and C1 all demonstrate an increased dose-corrected exposure relative to the reference compositions.

### DESCRIPTION

An aspect of the invention relates to a composition comprising a GLP-1 agonist and an absorption enhancer or delivery agent. The composition may be in the form suitable for oral administration, such as a tablet, sachet or capsule. In an embodiment the composition is an oral composition, or a pharmaceutical composition, such as an oral pharmaceutical composition.

The composition according to the invention in an embodiment includes a high content of the delivery agent and a minimal content of further excipients as described herein below. The provided compositions display an accelerated dissolution and absorption, enabling fast and efficient uptake of the active pharmaceutical ingredient.

### GLP-1

The term “GLP-1 agonist” as used herein refers to a compound, which fully or partially activates the human GLP-1 receptor. The term is thus equal to the term “GLP-1 receptor agonist” used in other documents. The term GLP-1 agonist as well as the specific GLP-1 agonists described herein are meant to encompass also salt forms hereof.

It follows that the GLP-1 agonist should display “GLP-1 activity” which refers to the ability of the compound, i.e. a GLP-1 analogue or a compound comprising a GLP-1 analogue, to bind to the GLP-1 receptor and initiate a signal transduction pathway resulting in insulinotropic action or other physiological effects as is known in the art. In some embodiments the “GLP-1 agonist” binds to a GLP-1 receptor, e.g., with an affinity constant (KD) or activate the receptor with a potency (EC₅₀) of below 1 μM, e.g. below 100 nM as measured by methods known in the art (see e.g. WO 98/08871) and exhibits insulinotropic activity, where insulinotropic activity may be measured in vivo or in vitro assays known to those of ordinary skill in the art. For example, the GLP-1 agonist may be administered to an animal with increased blood glucose (e.g. obtained using an Intravenous Glucose Tolerance Test (IVGTT). A person skilled in the art will be able to determine a suitable glucose dosage and a suitable blood sampling regime, e.g. depending on the species of the animal, for the IVGTT) and measure the plasma insulin concentration over time.

Suitable assays have been described in such as WO2015/155151.

The term half maximal effective concentration (EC₅₀) generally refers to the concentration which induces a response halfway between the baseline and maximum, by reference to the dose response curve. EC₅₀ is used as a measure of the potency of a compound and represents the concentration where 50% of its maximal effect is observed. Due to the albumin binding effects of GLP-1 agonists comprising a substituent as described herein, it is important to pay attention to if the assay includes human serum albumin or not.

The in vitro potency of the GLP-1 agonist may be determined as described in 2015/155151, example 29 (without HSA) and the EC50 deterimined. The lower the EC₅₀ value, the better the potency. In one embodiment the potency (EC50) as determined (without HSA) is 5-1000 pM, such as 10-750 pM, 10-500 pM or 10-200 pM. In one embodiment the EC50 (without HSA) is at most 500 pM, such as at most 300 pM, such as at most 200 pM.

In one embodiment the EC50 (without HSA) is comparable to human GLP-1(7-37).

In one embodiment the EC50 (without HSA) is at most 50 pM. In a further such embodiment the EC50 is at most 40 pM, such as at most 30 pM such as at most 20 pM, such as at most 10 pM. In one embodiment the EC50 is around 10 pM.

If desired, the fold variation in relation to a known GLP-1 receptor agonist may be calculated as EC50(test analogue)/EC50(known analogue), and if this ratio is such as 0.5-1.5, or 0.8-1.2 the potencies are considered to be equivalent.

In one embodiment the potency, EC50 (without HSA), is equivalent to the potency of liraglutide.

In one embodiment the potency, EC50 (without HSA), is equivalent to the potency of semaglutide.

In one embodiment the potency, EC50 (without HSA), is equivalent to the potency of Compound B.

In one embodiment the potency, EC50 (without HSA), is equivalent to the potency of Compound C.

In some embodiments the GLP-1 agonist is a GLP-1 analogue, optionally comprising one substituent. The term “analogue” as used herein referring to a GLP-1 peptide (hereafter “peptide”) means a peptide wherein at least one amino acid residue of the peptide has been substituted with another amino acid residue and/or wherein at least one amino acid residue has been deleted from the peptide and/or wherein at least one amino acid residue has been added to the peptide and/or wherein at least one amino acid residue of the peptide has been modified. Such addition or deletion of amino acid residues may take place at the N-terminal of the peptide and/or at the C-terminal of the peptide. In some embodiments a simple nomenclature is used to describe the GLP-1 agonist, e.g., [Aib8] GLP-1(7-37) designates an analogue of GLP-1(7-37) wherein the naturally occurring Ala in position 8 has been substituted with Aib. In some embodiments the GLP-1 agonist comprises a maximum of twelve, such as a maximum of 10, 8 or 6, amino acids which have been altered, e.g., by substitution, deletion, insertion and/or modification, compared to e.g. GLP-1(7-37). In some embodiments the analogue comprises up to 10 substitutions, deletions, additions and/or insertions, such as up to 9 substitutions, deletions, additions and/or insertions, up to 8 substitutions, deletions, additions and/or insertions, up to 7 substitutions, deletions, additions and/or insertions, up to 6 substitutions, deletions, additions and/or insertions, up to 5 substitutions, deletions, additions and/or insertions, up to 4 substitutions, deletions, additions and/or insertions or up to 3 substitutions, deletions, additions and/or insertions, compared to e.g. GLP-1(7-37). Unless otherwise stated the GLP-1 comprises only L-amino acids.

In some embodiments the term “GLP-1 analogue” or “analogue of GLP-1” as used herein refers to a peptide, or a compound, which is a variant of the human Glucagon-Like Peptide-1 (GLP-1(7-37)). GLP-1(7-37) has the sequence HAEGTFTSDV SSYLEGQAAKEFIAWLVKGRG (SEQ ID No: 1). In some embodiments the term “variant” refers to a compound which comprises one or more amino acid substitutions, deletions, additions and/or insertions.

In one embodiment the GLP-1 agonist exhibits at least 60%, 65%, 70%, 80% or 90% sequence identity to GLP-1(7-37) over the entire length of GLP-1(7-37). As an example of a method for determination of sequence identity between two analogues the two peptides [Aib8]GLP-1(7-37) and GLP-1(7-37) are aligned. The sequence identity of [Aib8]GLP-1(7-37) relative to GLP-1(7-37) is given by the number of aligned identical residues minus the number of different residues divided by the total number of residues in GLP-1(7-37). Accordingly, in said example the sequence identity is (31-1)/31.

In one embodiment the C-terminal of the GLP-1 agonist is an amide.

In some embodiments the GLP-1 agonist is GLP-1(7-37) or GLP-1(7-36)amide. In some embodiments the GLP-1 agonist is exendin-4, the sequence of which is HGEGTFITSDLSKQMEEEAVRLFIEWLKNGGPSSGAPPPS (SEQ ID No: 2).

In order to prolong the effect of the GLP-1 agonist it is preferred that the GLP-1 agonist have an extended half-life. The half-life can be determined by method known in the art and in an appropriate model, such as in Male Sprague Dawley rats or minipigs as described in WO2012/140117.

In one embodiment the GLP-1 agonist according to the invention has a half-life above 24 hours in minipig. In one embodiment the GLP-1 agonist according to the invention has a half-life above 30 hours, such as above 36 hours, such as above 42 hours, such as above 48 hours, such as above 54 hours or such as above 60 hours in minipig.

In some embodiments the GLP-1 agonist comprises one substituent which is covalently attached to the peptide. In some embodiments the substituent comprises a fatty acid or a fatty diacid. In some embodiments the substituent comprises a C16, C18 or C20 fatty acid. In some embodiments the substituent comprises a C16, C18 or C20 fatty diacid.

In some embodiments the substituent comprises formula (X)

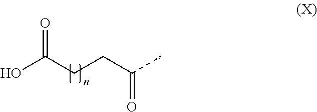

wherein n is at least 13, such as n is 13, 14, 15, 16, 17, 18 or 19. In some embodiments the substituent comprises formula (X), wherein n is in the range of 13 to 19, such as in the range of 13 to 17. In some embodiments the substituent comprises formula (X), wherein n is 13, 15 or 17. In some embodiments the substituent comprises formula (X), wherein n is 13. In some embodiments the substituent comprises formula (X), wherein n is 15. In some embodiments the substituent comprises formula (X), wherein n is 17.

In some embodiments the substituent comprises formula (XIa) HOOC—(C₆H₄)—O—(CH₂)ₘ—CO-* (XIa), wherein m is an integer in the range of 6-14

In some embodiments the substituent comprises formula (XIb)

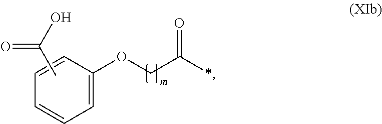

wherein the carboxy group is in position 2, 3 or 4 of the (C₆H₄) group and wherein m is an integer in the range of 8-11.

In some embodiments the substituent comprises formula (XIa) or formula (XIb), wherein m is in the range of 6 to 14, such as in the range of 8 to 11. In some embodiments the substituent comprises formula (XIa) or formula (XIb), wherein m is 8, 10 or 12. In some embodiments the substituent comprises formula (XIa) or formula (XIb), wherein m is 9. In some embodiments the substituent comprises formula (XIa) or formula (XIb), wherein m is 11.

In some embodiments the substituent comprises one or more 8-amino-3,6-dioxaoctanoic acid (OEG), such as two OEG.

In some embodiments the substituent is [2-(2-{2-[2-(2-{2-[(S)-4-carboxy-4-(17-carboxyheptadecanoylamino) butyrylamino]ethoxy}ethoxy)acetylamino] ethoxy}ethoxy)acetyl].

In some embodiments the substituent is [2-(2-{2-[2-(2-{2-[(S)-4-carboxy-4-({trans-4-[(19-carboxynonadecanoylamino)methyl]cyclohexanecarbonyl} amino)butyrylamino]ethoxy}ethoxy)acetylamino]ethoxy}ethoxy)acetyl].

In some embodiments the GLP-1 agonist is semaglutide, also known as N-epsilon26-[2-(2-{2-[2-(2-{2-[(S)-4-carboxy-4-(17-carboxyheptadecanoylamino) butyrylamino]ethoxy}ethoxy)acetylamino]ethoxy}ethoxy)acetyl][Aib8,Arg34]GLP-1(7-37) (SEQ ID NO. 4) which may be prepared as described in WO2006/097537, Example 4 with the following structure:

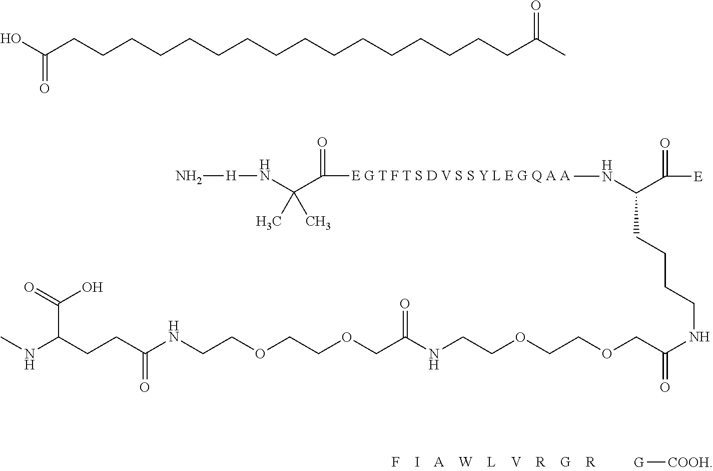

In one embodiment the GLP-1 agonist is GLP-1 agonist B, which is diacylated [Aib8,Arg34,Lys37]GLP-1(7-37) (SEQ ID NO. 5) as shown in Example 2 of WO2011/080103 and named N^{ϵ26} {2-[2-(2-{2-[2-{2{(S)-4-Carboxy-4-[10-(4-carboxyphenoxy)decanoylamino]butyrylamino}-ethoxy)ethoxy]acetylamino}ethoxy)ethoxy]acetyl}, N^{ϵ37}-{2-[2-(2-{2-[2-(2-{(S)-4-carboxy-4-[10-(4-carboxyphenoxy)decanoylamino]butyrylamino}ethoxy)ethoxy]acetylamino}ethoxy)ethoxy]-acetyl}-[Aib⁸,Arg³⁴,Lys³⁷]GLP-1(7-37)-peptide with the following structure.

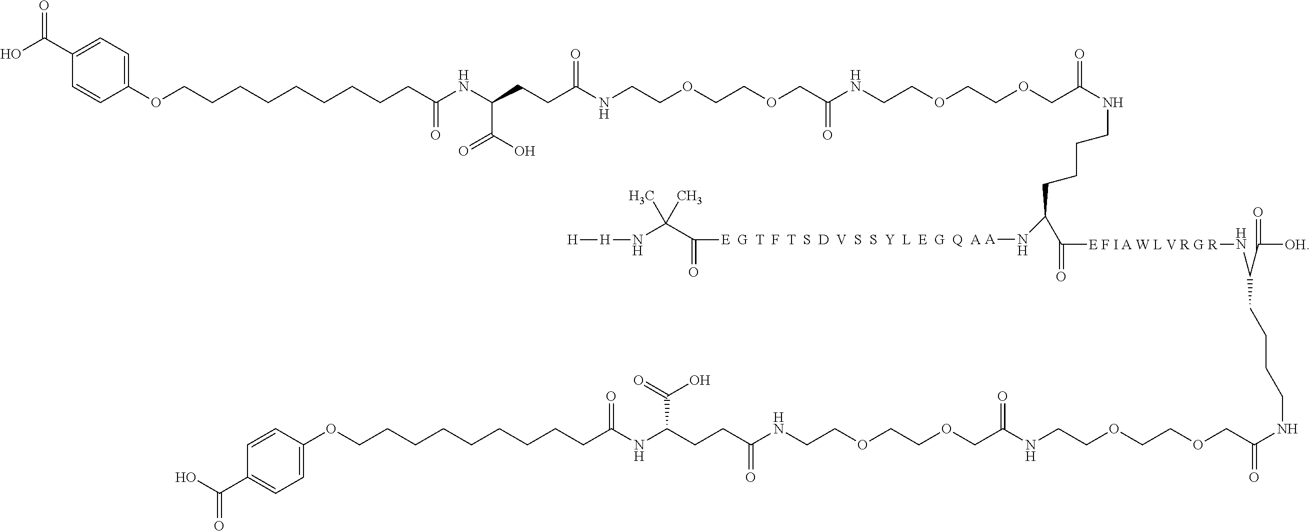

In one embodiment the GLP-1 agonist is GLP-1 agonist C which is Diacylated [Aib8,Glu22,Arg26,Lys27,Glu30,Arg34,Lys36]-GLP-1-(7-37)-peptidyl-Glu-Gly (SEQ ID NO. 6) as shown in Example 31 of WO2012/140117 and named N^{ϵ27}-[2-[2-[2-[[2-[2-[2-[[(4S)-4-carboxy-4-[10-(4- carboxyphenoxy)decanoylamino]butanoyl]amino] ethoxy]ethoxy]acetyl]amino]ethoxy]ethoxy]-acetyl], N^{ϵ36}-[2-[2-[2-[[2-[2-[2-[[(4S)-4-carboxy-4-[10-(4- carboxyphenoxy)decanoylamino]-butanoyl]amino]ethoxy]ethoxy]acetyl]amino]ethoxy]ethoxy]acetyl]-[Aib8,Glu22,Arg26,Lys27, Glu30,Arg34,Lys36]-GLP-1-(7-37)-peptidyl-Glu-Gly with the following structure

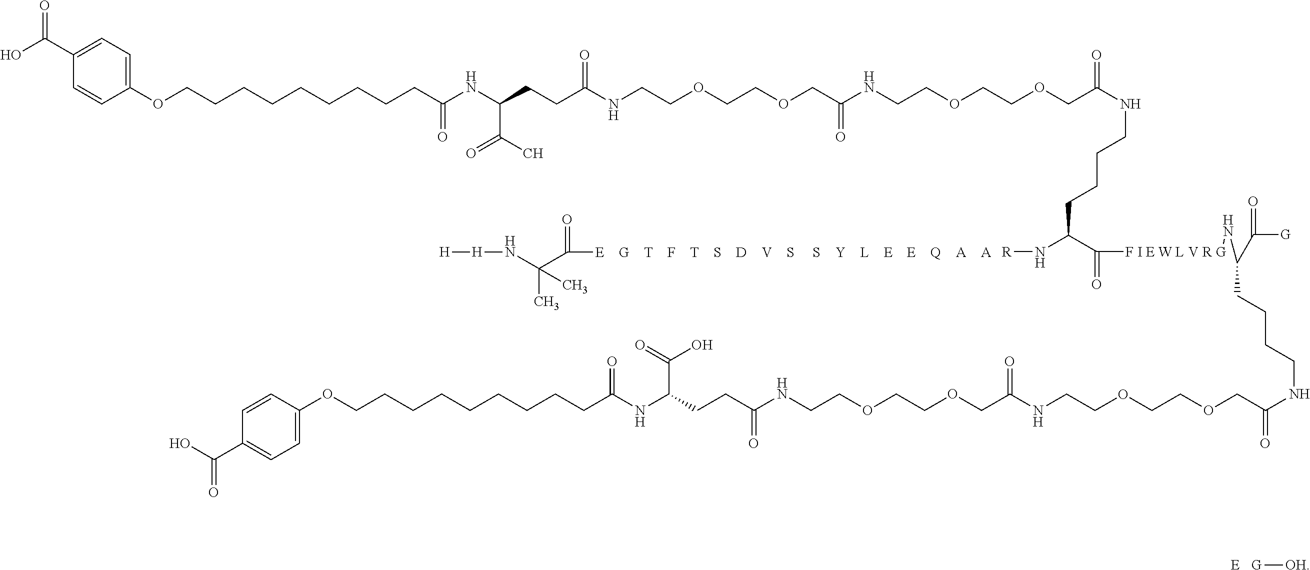

In general the term GLP-1 agonist is meant to encompass the GLP-1 agonist and any pharmaceutically acceptable salt, amide, or ester thereof. In some embodiments the composition comprises the GLP-1 agonist or a pharmaceutically acceptable salt, amide, or ester thereof. In some embodiments the composition comprises the GLP-1 agonist and one or more pharmaceutically acceptable counter ions.

In some embodiments the GLP-1 agonist is selected from one or more of the GLP-1 agonists mentioned in WO93/19175, WO96/29342, WO98/08871, WO99/43707, WO99/43706, WO99/43341, WO99/43708, WO2005/027978, WO2005/058954, WO2005/058958, WO2006/005667, WO2006/037810, WO2006/037811, WO2006/097537, WO2006/097538, WO2008/023050, WO2009/030738, WO2009/030771 and WO2009/030774.

In some embodiments the GLP-1 agonist is selected from the group consisting of N-epsilon37{2-[2-(2-{2-[2-((R)-3-carboxy-3-{[1-(19-carboxynonadecanoyl) piperidine-4-carbonyl]amino}propionylamino)ethoxy]ethoxy}acetylamino)ethoxy]ethoxy}acetyl [desaminoHis7,Glu22,Arg26,Arg34,Lys37]GLP-1(7-37)amide; N-epsilon26{2-[2-(2-{2-[2-((R)-3-carboxy-3-{[1-(19- carboxynonadecanoyl) piperidine-4-carbonyl]amino} propionylamino)ethoxy]ethoxy}acetylamino)ethoxy] ethoxy}acetyl [desaminoHis7, Arg34] GLP-1-(7-37); N-epsilon37{2-[2-(2-{2-[2-((S)-3-carboxy-3-{[1-(19-carboxy-nonadecanoyl) piperidine-4-carbonyl]amino}propionylamino)ethoxy] ethoxy} acetylamino)ethoxy] ethoxy}acetyl[Aib8,Glu22,Arg26,Arg34,Lys37]GLP-1-(7-37)amide; N-epsilon37-[2-(2-[2-(2-[2-(2-((R)-3-[1-(17- carboxyheptadecanoyl)piperidin-4-ylcarbonylamino]3-carboxypropionylamino)ethoxy)ethoxy]acetylamino)ethoxy] ethoxy)acetyl][,DesaminoHis7, Glu22 Arg26, Arg 34, Phe(m-CF3)28]GLP-1-(7-37)amide; N-epsilon26-[(S)-4-carboxy-4-({trans-4-[(19-carboxynonadecanoylamino)methyl] cyclohexanecarbonyl}amino)butyryl][Aib8,Arg34]GLP-1-(7-37); N-epsilon26-{4-[(S)-4-carboxy-4-({trans-4-[(19-carboxynonadecanoylamino) methyl]cyclohexanecarbonyl} amino)butyrylamino]butyryl}[Aib8,Arg34]GLP-1-(7-37); N-epsilon26-[2-(2-{2-[(S)-4-carboxy-4-({trans-4-[(19-carboxy- nonadecanoylamino) methyl]cyclohexanecarbonyl} amino)butyrylamino]ethoxy}ethoxy)acetyl][Aib8,Arg34]GLP-1-(7-37); N-epsilon26-[2-(2-{2-[2-(2-{2-[(S)-4-carboxy-4-({trans-4-[(19-carboxy-nonadecanoylamino)methyl] cyclohexanecarbonyl}amino)butyrylamino]ethoxy}ethoxy)acetylamino]ethoxy}ethoxy) acetyl][Aib8,Arg34]GLP-1-(7-37)amide; N-epsilon37-[2-(2-{2-[2-(2-{2-[(S)-4-carboxy-4-({trans-4-[(19-carboxy- nonadecanoylamino)methyl]cyclohexanecarbonyl}amino) butyrylamino]ethoxy}ethoxy)acetylamino]ethoxy}ethoxy)acetyl][Aib8,Glu22,Arg26, Arg34,Lys37]GLP-1-(7-37)amide; N-epsilon37-[2-(2-{2-[2-(2-{2-[(S)-4-carboxy-4-({trans-4-[(19-carboxy- nonadecanoylamino)methyl]cyclohexanecarbonyl}amino) butyrylamino] ethoxy}ethoxy)acetylamino]ethoxy}ethoxy)acetyl][DesaminoHis7,Glu22, Arg26,Arg34,Lys37]GLP-1-(7-37)amide; N-epsilon37-[2-(2-{2-[2-(2-{2-[(S)-4-carboxy-4-({4-[(trans-19-carboxy- nonadecanoylamino)methyl]cyclohexanecarbonyl}amino) butyrylamino]ethoxy}ethoxy)acetylamino]ethoxy}ethoxy)acetyl][DesaminoHis7,Arg26,Arg34,Lys37]GLP-1-(7-37)amide; N-epsilon37-[2-(2-{2-[2-(2-{2-[(S)-4-carboxy-4-({trans-4-[(19-carboxy-nonadecanoylamino)methyl]cyclohexanecarbonyl}amino) butyrylamino]ethoxy}ethoxy)acetylamino]ethoxy}ethoxy)acetyl][DesaminoHis7,Glu22,Arg26,Arg34,Lys37]GLP-1-(7-37); N-epsilon26[2-(2-{2-[2-(2-{2-[(S)-4-carboxy-4-({4-[(19-carboxy-nonadecanoylamino)methyl]cyclohexanecarbonyl}amino)butyrylamino] ethoxy}ethoxy) acetylamino]ethoxy}ethoxy)acetyl[Aib8, Lys 26]GLP-1 (7-37)amide; N-epsilon26 [2-(2-[2-(2-[2-(2-((S)-2-[trans-4-((9- carboxynonadecanoylamino] methyl) cyclohexylcarbonylamino]-4-carboxybutanoylamino)ethoxy)ethoxy]acetylamino) ethoxy]ethoxy)acetyl][Aib8, Lys26] GLP-1 (7-37)amide; N-epsilon37-[2-(2-{2-[2-(2-{2-[(S)-4-carboxy-4-({trans-4-[(19-carboxy-nonadecanoylamino)methyl]cyclohexane- carbonyl} amino)butyrylamino]ethoxy}ethoxy)acetylamino]ethoxy}ethoxy)acetyl] [DesaminoHis7,Arg26,Arg34,Lys37]GLP-1-(7-37); N-epsilon37-[2-(2-{2-[2-(2-{2-[(S)-4-carboxy-4-({trans-4-[(19-carboxy-nonadecanoylamino)methyl]cyclohexanecarbonyl} amino)butyrylamino]ethoxy}ethoxy)acetylamino]ethoxy}ethoxy)acetyl][DesaminoHis7,Glu22, Arg26,Glu30,Arg34,Lys37]GLP-1-(7-37); N-epsilon26-[2-(2-{2-[(S)-4-carboxy-4-((S)-4-carboxy-4-{4-[4-(16-(1H-tetrazol-5-yl)-hexadecanoylsulfamoyl)butyrylamino]-butyrylamino}butyrylamino) butyrylamino] ethoxy}ethoxy)acetyl][Aib8,Arg34]GLP-1-(7-37); N-epsilon26-[2-(2-{2-[(S)-4-carboxy-4-((S)-4-carboxy-4-{12-[4-(16-(1H-tetrazol-5-yl)hexadecanoyl-sulfamoyl)butyrylamino]dodecanoylamino}butyrylamino) butyrylamino]ethoxy}ethoxy)acetyl][Aib8,Arg34]GLP-1-(7-37); N-epsilon26-[2-(2-{2-[(S)-4-carboxy-4-((S)-4-carboxy-4-{6-[4-(16-(1H-tetrazol-5-yl)hexadecanoyl-sulfamoyl)butyrylamino]hexanoylamino} butyrylamino)butyrylamino]ethoxy}ethoxy) acetyl][Aib8,Arg34]GLP-1-(7-37); N-epsilon26-[2-(2-{2-[(S)-4-carboxy-4-((S)-4-carboxy-4-{4-[4-(16-(1H- tetrazol-5-yl)hexadecanoylsulfamoyl)butyrylamino] butyrylamino}butyrylamino)butyrylamino]ethoxy}ethoxy)acetyl][Aib8,Arg34]GLP-1-(7-34); N-epsilon26-[2-(2-{2-[(S)-4-carboxy-4-((S)-4- carboxy-4-{12-[4-(16-(1H-tetrazol-5-yl)hexadecanoylsulfamoyl)butyrylamino]-dodecanoylamino}butyrylamino) butyrylamino] ethoxy}ethoxy)acetyl][Aib8,Arg34]GLP-1-(7-34); N-epsilon26-[2-(2-{2-[(S)-4-carboxy-4-((S)-4-carboxy-4-{6-[4-(16- (1H-tetrazol-5-yl)hexadecanoylsulfamoyl) butyrylamino]hexanoylamino}butyrylamino) butyrylamino]ethoxy}ethoxy)acetyl] [Aib8,Arg34]GLP-1-(7-34); N-epsilon26-[2-(2-{12-[(S)-4-carboxy-4-((S)-4-carboxy-4-{12-[4-(16-(1H-tetrazol-5-yl)hexadecanoyl-sulfamoyl)butyrylamino]dodecanoylamino} butyrylamino)butyrylamino]ethoxy}ethoxy)acetyl][Aib8,Arg34]GLP-1-(7-35); N-epsilon26-[2-(2-{2-[(S)-4-carboxy-4-((S)-4-carboxy-4-{6-[4-(16-(1H-tetrazol-5-yl)hexadecanoylsulfamoyl)butyrylamino]hexanoylamino} butyrylamino)butyrylamino] ethoxy}ethoxy)acetyl][Aib8,Arg34]GLP-1-(7-35); N-epsilon26-[2-(2-{2-[(S)-4-carboxy-4-((S)-4-carboxy-4-{6-[4-(16- (1H-tetrazol-5-yl)hexadecanoylsulfamoyl)butyrylamino] hexanoylamino}butyrylamino)butyrylamino]ethoxy}ethoxy)acetyl][Aib8,Arg34]GLP-1-(7-36)amide; N-epsilon26-[2-(2-{2-[(S)-4-carboxy-4-((S)-4-carboxy-4-{6-[4-(16-(1H-tetrazol-5-yl)hexadecanoylsulfamoyl) butyrylamino]hexanoylamino}butyrylamino) butyrylamino]ethoxy}ethoxy)acetyl][Aib8,Arg34]GLP-1-(7-35); N-epsilon26-[2-(2-{2-[(S)-4-carboxy-4-((S)-4-carboxy-4-{12-[4-(16-(1H-tetrazol-5-yl)hexadecanoyl-sulfamoyl)butyrylamino]dodecanoylamino}butyryl-amino)butyrylamino]ethoxy} ethoxy)acetyl][Aib8,Lys33,Arg34]GLP-1-(7-34); N-epsilon26-[2-(2-{12-[(S)-4-carboxy-4-((S)-4-carboxy-4-{12-[4-(16-(1H-tetrazol-5-yl)hexadecanoylsulfamoyl)butyrylamino] dodecanoylamino}butyrylamino)butyrylamino]ethoxy}ethoxy)acetyl][Aib8,Arg34]GLP-1-(7-36)amide; N-epsilon26-[2-(2-{2-[2-(2-{2-[2-(2-{2-[2-(2-{2-[2-(2-{2-[2-(2-{2-[(S)-4- carboxy-4-((S)-4-carboxy-4-{12-[4-(16-(1H-tetrazol-5-yl)hexadecanoylsulfamoyl) butyrylamino]dodecanoylamino}butyrylamino) butyrylamino]ethoxy}ethoxy) acetylamino]ethoxy}ethoxy)acetylamino]ethoxy}ethoxy)acetylamino]ethoxy}ethoxy)acetylamino]ethoxy}ethoxy)acetylamino]ethoxy}ethoxy)acetyl][Aib8,Lys26,Arg34]GLP-1-(7-36)amide; N-epsilon37-[2-(2-{2-[(S)-4-carboxy-4-((S)-4-carboxy-4-{12-[4-(16-(1H-tetrazol-5-yl)hexadecanoylsulfamoyl)butyrylamino] dodecanoylamino}butyrylamino) butyrylamino]ethoxy}ethoxy)acetyl][Aib8,Glu22,Arg26,Arg34,Lys37]GLP-1-(7-37)amide; N-epsilon37-[2-(2-{2-[(S)-4-carboxy-4-((S)- 4-carboxy-4-{12-[4-(16-(1H-tetrazol-5-yl)hexadecanoylsulfamoyl)butyrylamino]dodecanoylamino}butyrylamino) butyrylamino] ethoxy}ethoxy)acetyl][DesaminoHis7,Glu22,Arg26,Arg34,Lys37]GLP-1-(7-37)amide; N-epsilon37{2-[2-(2-{2-[2-((R)-3-carboxy-3-{[1-(19-carboxy-nonadecanoyl) piperidine-4-carbonyl]amino}propionylamino)ethoxy]ethoxy} acetylamino)ethoxy] ethoxy}acetyl [desaminoHis7,Glu22,Arg26,Arg34,Lys37]GLP-1(7-37)amide; N-epsilon37{2-[2-(2-{2-[2-((S)-3-carboxy-3-{[1-(19- carboxynonadecanoyl) piperidine-4-carbonyl]amino} propionylamino) ethoxy]ethoxy}acetylamino)ethoxy] ethoxy} acetyl [Aib8,Glu22, Arg26, Arg34, Lys37]GLP-1-(7-37)amide; N-epsilon37-[2-(2-[2-(2-[2-(2-((R)-3-[1-(17-carboxyhepta-decanoyl)piperidin-4-ylcarbonylamino]3-carboxy-propionylamino) ethoxy)ethoxy] acetylamino) ethoxy] ethoxy)acetyl] [DesaminoHis7, Glu22,Arg26, Arg34,Phe(m-CF3)28] GLP-1-(7-37)amide; N-epsilon37-[2-(2-{2-[2-(2-{2- [(S)-4-carboxy-4-({trans-4-[(19-carboxy-nonadecanoylamino)methyl] cyclohexanecarbonyl} amino)butyrylamino]ethoxy} ethoxy)acetylamino] ethoxy}ethoxy)acetyl] [Aib8,Glu22,Arg26,Arg34,Lys37]GLP-1-(7-37)amide; N-epsilon37-[2-(2-{2-[2-(2-{2-[(S)-4-carboxy-4-({trans-4-[(19-carboxy-nonadecanoylamino)methyl]cyclohexane-carbonyl} amino)butyrylamino]ethoxy}ethoxy) acetylamino]ethoxy}ethoxy)acetyl] [DesaminoHis7,Glu22,Arg26,Arg34,Lys37]GLP-1-(7-37)amide; N-epsilon37-[2-(2-{2-[2-(2-{2-[(S)-4-carboxy-4-({trans-4-[(19-carboxy-nonadecanoylamino)methyl] cyclohexanecarbonyl}amino)butyrylamino]ethoxy}ethoxy) acetylamino]ethoxy} ethoxy)acetyl] [DesaminoHis7,Glu22,Arg26,Arg34, Lys37]GLP-1-(7-37); N-epsilon37-[2-(2-{2-[2-(2-{2-[(S)-4-carboxy-4-({trans-4-[(19-carboxy-nonadecanoylamino) methyl]cyclohexane-carbonyl}amino)butyrylamino]ethoxy}ethoxy) acetylamino] ethoxy}ethoxy)acetyl] [DesaminoHis7,Glu22,Arg26,Glu30,Arg34, Lys37]GLP-1-(7-37); N-epsilon37-[2-(2-{2-[(S)-4-carboxy-4- ((S)-4-carboxy-4-{12-[4-(16-(1H-tetrazol-5-yl)hexadecanoyl-sulfamoyl) butyrylamino]dodecanoylamino} butyrylamino) butyrylamino] ethoxy}ethoxy)acetyl] [Aib8,Glu22,Arg26,Arg34,Lys37]GLP-1-(7-37)amide; N-epsilon37-[2-(2-{2-[(S)-4-carboxy-4-((S)-4- carboxy-4-{12-[4-(16-(1H-tetrazol-5-yl)hexadecanoylsulfamoyl) butyrylamino]dodecanoylamino}butyrylamino) butyrylamino] ethoxy}ethoxy)acetyl] [DesaminoHis7,Glu22,Arg26,Arg34,Lys37]GLP-1-(7-37)amide; N-epsilon37-(3-((2-(2-(2-(2-(2-Hexadecyloxyethoxy)ethoxy)ethoxy) ethoxy) ethoxy)) propionyl)[DesaminoHis7,Glu22,Arg26,Arg34,Lys37]GLP-1(7-37)-amide; N-epsilon37-{2-(2-(2-(2-[2-(2-(4-(hexadecanoylamino)-4-carboxybutyryl-amino)ethoxy) ethoxy] acetyl)ethoxy)ethoxy)acetyl)}-[desaminoHis7,Glu22,Arg26, Glu30,Arg34,Lys37] GLP-1-(7-37)amide; N-epsilon37-{2-(2-(2-(2-[2-(2-(4-(hexadecanoylamino)-4-carboxy-butyryl-amino) ethoxy)ethoxy]acetyl)ethoxy)ethoxy) acetyl)[desaminoHis7,Glu22, Arg26, Arg34,Lys37]GLP-1-(7-37)amide; N-epsilon37-(2-(2-(2-(2-(2-(2-(2-(2-(2-(octadecanoyl-amino)ethoxy)ethoxy) acetylamino)ethoxy) ethoxy)acetylamino) ethoxy)ethoxy) acetyl)[desaminoHis7,Glu22,Arg26,Arg34,Lys37] GLP-1 (7-37)amide; N-epsilon37-[4-(16-(1H-Tetrazol-5-yl)hexadecanoylsulfamoyl) butyryl] [DesaminoHis7,Glu22,Arg26, Arg34, Lys37]GLP-1-(7-37)amide; N-epsilon37-[2-(2-{2-[2-(2-{2-[(S)-4-carboxy-4-(19-carboxynonadecanoylamino) butyrylamino] ethoxy}ethoxy) acetylamino]ethoxy} ethoxy)acetyl] [DesaminoHis7,Glu22,Arg26, Arg34,Lys37]GLP-1-(7-37); N-epsilon37-(2-{2-[2-((S)-4-carboxy-4-{(S)-4-carboxy-4-[(S)-4-carboxy-4-(19-carboxy-nonadecanoylamino)butyrylamino]butyrylamino} butyrylamino)ethoxy]ethoxy} acetyl)[DesaminoHis7,Glu22,Arg26,Arg34,Lys37]GLP-1-(7-37); N-epsilon37-{2-[2-(2-{(S)-4-[(S)-4-(12-{4-[16-(2-tert-Butyl-2H-tetrazol-5-yl)-hexadecanoylsulfamoyl] butyrylamino}dodecanoylamino)-4-carboxybutyrylamino]-4-carboxybutyrylamino} ethoxy)ethoxy]acetyl}[DesaminoHis7,Glu22,Arg26,Arg34,Lys37] GLP-1 (7-37); N-epsilon37-[2-(2-12-[2-(2-{2-[(S)-4- carboxy-4-(17-carboxy-heptadecanoylamino)-butyrylamino]-ethoxy}-ethoxy)-acetylamino]-ethoxy}-ethoxy)-acetyl] [Aib8,Glu22, Arg26,Arg34,Lys37]GLP-1-(7-37); N-alpha37-[2-(2-{2-[2-(2-{2-[(S)-4-carboxy-4-(17-carboxy-heptadecanoylamino)-butyrylamino]-ethoxy}-ethoxy)-acetylamino]-ethoxy}-ethoxy)-acetyl] [Aib8,Glu22,Arg26,Arg34,epsilon-Lys37]GLP-1-(7-37)peptide; N-epsilon37-[2-(2-{2-[2-(2-{2-[(S)-4-carboxy-4-(17-carboxy-heptadecanoylamino)-butyrylamino]-ethoxy}-ethoxy)-acetylamino]-ethoxy}-ethoxy)-acetyl] [desaminoHis7, Glu22,Arg26,Arg34,Lys37] GLP-1-(7-37); N-epsilon36-[2-(2-{2-[2-(2-{2-[(S)-4-carboxy-4-(15-carboxy-pentadecanoylamino)-butyrylamino]-ethoxy}-ethoxy)-acetylamino]-ethoxy}-ethoxy)-acetyl] [desaminoHis7, Glu22,Arg26,Glu30,Arg34,Lys36] GLP-1-(7-37)-Glu-Lys peptide; N-epsilon37-[2-(2-{2-[2-(2-{2-[(S)-4-carboxy-4- ({trans-4-[(19-carboxynonadecanoylamino)methyl]cyclohexanecarbonyl}amino)butyryl-amino]ethoxy}ethoxy)acetylamino]ethoxy}ethoxy)acetyl][Aib8,Glu22,Arg26,Arg34,Lys37]GLP-1-(7-37); N-epsilon37-[2-(2-{2-[2-(2-{2-[(S)-4-carboxy-4-(17-carboxy-heptadecanoylamino)-butyrylamino]-ethoxy}-ethoxy)-acetylamino]-ethoxy}-ethoxy)- acetylHAib8,Glu22, Arg26,Arg34,Aib35,Lys37]GLP-1-(7-37); N-epsilon37-[(S)-4-carboxy-4-(2-{2-[2-(2-{2-[2-(17- carboxyheptadecanoylamino) ethoxy] ethoxy} acetylamino) ethoxy] ethoxy} acetylamino) butyryl] [Aib8,Glu22,Arg26,34,Lys37] GLP-1 (7-37); N-epsilon37-[2-(2-[2-(2-[2-(2-[4-(17-carboxyheptadecanoylamino)- 4(S)-carboxybutyry-lamino]ethoxy)ethoxy]acetylamino)ethoxy]ethoxy)acetyl] [ImPr7,Glu22, Arg26,34,Lys37], GLP-1-(7-37); N-epsilon26-{2-[2-(2-{2-[2-(2-{(S)-4-carboxy-4-[10-(4-carboxyphenoxy) decanoylamino]butyrylamino}ethoxy)ethoxy] acetylamino}ethoxy) ethoxy]acetyl}, N-epsilon37-{2-[2-(2-{2-[2-(2-{(S)-4-carboxy-4-[1 0-(4-carboxy-phenoxy) decanoylamino] butyrylamino}ethoxy)ethoxy]acetylamino}ethoxy) ethoxy]acetyl}-[Aib8,Arg34,Lys37]GLP-1(7-37)-OH; N-epsilon26 (17-carboxyhepta-decanoyl)-[Aib8,Arg34]GLP-1-(7-37)-peptide; N-epsilon26-(19-carboxynonadecanoyl)-[Aib8,Arg34]GLP-1-(7-37); N-epsilon26-(4-{[N-(2-carboxyethyl)-N-(15- carboxypenta-decanoyl)amino]methyl}benzoyl[Arg34]GLP-1-(7-37); N-epsilon26-[2-(2-[2-(2-[2-(2-[4-(17-carboxyheptadecanoylamino)-4(S)-carboxybutyrylamino]ethoxy)ethoxy] acetylamino) ethoxy]ethoxy)acetyl][Aib8,Arg34]GLP-1-(7-37); N-epsilon26-[2-(2-[2-(2-[2-(2-[4-(19-carboxynonadecanoylamino)-4(S)-carboxybutyrylamino]ethoxy)ethoxy] acetylamino)ethoxy]ethoxy)acetyl][Aib8,Arg34]GLP-1-(7-37); N-epsilon26-[2-(2-[2-(2-[2-(2-[4-(17-carboxyheptadecanoylamino)-4(S)-carboxybutyrylamino]ethoxy)ethoxy] acetylamino)ethoxy]ethoxy)acetyl][3-(4-Imidazolyl)Propionyl7,Arg34]GLP-1-(7-37); N-epsilon26-[2-(2-[2-(2-[2-(2-[4-(17-carboxyheptadecanoylamino)-(carboxymethyl- amino)acetylamino]ethoxy)ethoxy]acetylamino)ethoxy]ethoxy)acetyl][Aib8,Arg34]GLP-1-(7-37); N-epsilon26-[2-(2-[2-(2-[2-(2-[4-(17-carboxyheptadecanoylamino)-3(S)-Sulfopropionylamino]ethoxy)ethoxy]acetylamino)ethoxy]ethoxy)acetyl][Aib8,Arg34]GLP-1-(7-37); N-epsilon26-[2-(2-[2-(2-[2-(2-[4-(17-carboxyheptadecanoylamino)-4(S)-carboxybutyrylamino]ethoxy)ethoxy]acetylamino)ethoxy]ethoxy)acetyl][Gly8,Arg34] GLP-1-(7-37); N-epsilon26-[2-(2-[2-(2-[2-(2-[4-(17-carboxyheptadecanoylamino)-4(S)-carboxybutyrylamino]ethoxy)ethoxy]acetylamino)ethoxy]ethoxy)acetyl][Aib8,Arg34]GLP-1-(7-37)-amide; N-epsilon26-[2-(2-[2-(2-[2-(2-[4-(17-carboxyheptadecanoylamino)-4(S)-carboxybutyrylamino]ethoxy)ethoxy]acetylamino)ethoxy]ethoxy)acetyl] [Aib8,Arg34,Pro37]GLP-1-(7-37)amide; Aib8,Lys26(N-epsilon26-{2-(2-(2-(2-[2-(2-(4-(pentadecanoylamino)-4- carboxybutyrylamino)ethoxy)ethoxy]acetyl)ethoxy) ethoxy)acetyl)}), Arg34)GLP-1 H(7-37)-OH; N-epsilon26-[2-(2-[2-(2-[2-(2-[4-{[N-(2-carboxyethyl)-N-(17-carboxyheptadecanoyl)amino]methyl}benzoyl)amino]ethoxy) ethoxy]acetylamino)ethoxy]ethoxy)acetyl][Aib8,Arg34]GLP-1(7-37); N-alpha7-formyl, N-epsilon26-[2-(2-[2-(2-[2-(2-[4-(17- carboxyheptadecanoyl-amino)-4(S)-carboxy-butyrylamino]ethoxy)ethoxy]acetylamino)ethoxy]ethoxy)acetyl] [Arg34]GLP-1-(7-37); N-epsilon2626-[2-(2-[2-(2-[2-(2-[4-(17-carboxy-heptadecanoylamino)-4(S)-carboxy-butyrylamino]ethoxy)ethoxy]acetylamino)ethoxy]ethoxy)acetyl][Aib8, Glu22, Arg34] GLP-1-(7-37); N-epsilon26{3-[2-(2-{2-[2-(2-{2-[2-(2-[4-(15-(N-((S)-1,3-dicarboxypropyl) carbamoyl)pentadecanoylamino)-(S)-4-carboxybutyrylamino] ethoxy)ethoxy] ethoxy}ethoxy)ethoxy]ethoxy}ethoxy)ethoxy]propionyl} [Aib8,Arg34]GLP-1-(7-37); N-epsilon26-[2-(2-[2-(2-[2-(2-[4-{[N-(2-carboxyethyl)-N-(17-carboxy-heptadecanoyl)amino]methyl}benzoyl)aminoy4(S)-carboxybutyryl-amino)ethoxy) ethoxy]acetylamino)ethoxy]ethoxy)acetyl][Aib8,Arg34] GLP-1(7-37); N-epsilon26-{(S)-4-carboxy-4-((S)-4-carboxy-4-((S)-4-carboxy-4-((S)-4- carboxy-4-(19-carboxy-nonadecanoylamino)butyrylamino)butyrylamino)butyrylamino) butyrylamino} [Aib8,Arg34]GLP-1-(7-37); N-epsilon26-4-(17-carboxyheptadecanoyl-amino)-4(S)-carboxybutyryl-[Aib8,Arg34]GLP-1-(7-37); N-epsilon26- {3-[2-(2-{2-[2-(2-{2-[2-(2-[4-(17-carboxyheptadecanoylamino)-4(S)-carboxybutyrylamino]ethoxy)ethoxy]ethoxy} ethoxy)ethoxy]ethoxy}ethoxy)ethoxy]propionyl}[Aib8,Arg34]GLP-1-(7-37); N-epsilon26-{2-(2-(2-(2-[2-(2-(4-(17-carboxyheptadecanoylamino)-4-carboxybutyrylamino) ethoxy)ethoxy]acetyl)ethoxy)ethoxy)acetyl)}-Aib8,22,27,30,35,Arg34,Pro37, Lys26] GLP-1 (7-37)amide; N-epsilon26-[2-(2-[2-[4-(21-carboxyuneicosanoylamino)-4(S)-carboxybutyrylamino]ethoxy]ethoxy)acetyl][Aib8,Arg34]GLP-1-(7-37); and N-epsilon26-[2-(2-[2-(2-[2-(2-[4-(21-carboxyuneicosanoylamino)-4(S)-carboxybutyrylamino] ethoxy)ethoxy]acetylamino)ethoxy]ethoxy)acetyl][Aib8,Arg34]GLP-1-(7-37).

### Delivery Agent

### Salt of N-(8-(2-Hydroxybenzoyl)Amino)Caprylic Acid

The delivery agent used in the present invention is a salt of N-(8-(2-hydroxybenzoyl)amino)caprylic acid (NAC). The structural formula of N-(8-(2-hydroxybenzoyl)amino)caprylate is shown in formula (I).

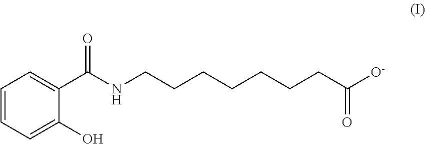 [SMILES: O=C([O-])CCCCCCCNC(=O)c1ccccc1O]

In some embodiments the salt of N-(8-(2-hydroxybenzoyl)amino)caprylic acid comprises one monovalent cation, two monovalent cations or one divalent cation. In some embodiments the salt of N-(8-(2-hydroxybenzoyl)amino)caprylic acid is selected from the group consisting of the sodium salt, potassium salt and/or calcium salt of N-(8-(2-hydroxybenzoyl)amino)caprylic acid. In one embodiment the salt of N-(8-(2-hydroxybenzoyl)amino)caprylic acid is selected from the group consisting of the sodium salt, potassium salt and/or the ammonium salt. In one embodiment the salt of N-(8-(2-hydroxybenzoyl)amino)caprylic acid is the sodium salt or the potassium salt. Salts of N-(8-(2-hydroxybenzoyl)amino)caprylate may be prepared using the method described in e.g. WO96/030036, WO00/046182, WO01/092206 or WO2008/028859.

The salt of N-(8-(2-hydroxybenzoyl)amino)caprylic acid may be crystalline and/or amorphous. In some embodiments the delivery agent comprises the anhydrate, monohydrate, dihydrate, trihydrate, a solvate or one third of a hydrate of the salt of N-(8-(2-hydroxybenzoyl)amino)caprylic acid as well as combinations thereof. In some embodiments the delivery agent is a salt of N-(8-(2-hydroxybenzoyl)amino)caprylic acid as described in WO2007/121318.

In some embodiments the delivery agent is sodium N-(8-(2-hydroxybenzoyl)amino)caprylate (referred to as “SNAC” herein), also known as sodium 8-(salicyloylamino)octanoate.

### Composition

The composition or pharmaceutical composition of the present invention is a solid or dry composition suited for administration by the oral route as described further herein below.

In some embodiments the composition comprises at least one pharmaceutically acceptable excipient. The term “excipient” as used herein broadly refers to any component other than the active therapeutic ingredient(s) or active pharmaceutical ingredient(s) (API(s)).

A excipient may be a pharmaceutically inert substance, an inactive substance, and/or a therapeutically or medicinally none active substance.

The excipients may serve various purposes, e.g. as a carrier, vehicle, filler, binder, lubricant, glidant, disintegrant, flow control agent, crystallization inhibitors, solubilizer, stabilizer, colouring agent, flavouring agent, surfactant, emulsifier or combinations of thereof and/or to improve administration, and/or absorption of the therapeutically active substance(s) or active pharmaceutical ingredient(s). The amount of each excipient used may vary within ranges conventional in the art. Techniques and excipients which may be used to formulate oral dosage forms are described in Handbook of Pharmaceutical Excipients, 8th edition, Sheskey et al., Eds., American Pharmaceuticals Association and the Pharmaceutical Press, publications department of the Royal Pharmaceutical Society of Great Britain (2017); and Remington: the Science and Practice of Pharmacy, 22nd edition, Remington and Allen, Eds., Pharmaceutical Press (2013).

In some embodiments the excipients may be selected from binders, such as polyvinyl pyrrolidone (povidone), etc.; fillers such as cellulose powder, microcrystalline cellulose, cellulose derivatives like hydroxymethylcellulose, hydroxyethylcellulose, hydroxypropylcellulose and hydroxy-propylmethylcellulose, dibasic calcium phosphate, corn starch, pregelatinized starch, etc.; lubricants and/or glidants such as stearic acid, magnesium stearate, sodium stearylfumarate, glycerol tribehenate, etc.; flow control agents such as colloidal silica, talc, etc.; crystallization inhibitors such as Povidone, etc.; solubilizers such as Pluronic, Povidone, etc.; colouring agents, including dyes and pigments such as iron oxide red or yellow, titanium dioxide, talc, etc.; pH control agents such as citric acid, tartaric acid, fumaric acid, sodium citrate, dibasic calcium phosphate, dibasic sodium phosphate, etc.; surfactants and emulsifiers such as Pluronic, polyethylene glycols, sodium carboxymethyl cellulose, polyethoxylated and hydrogenated castor oil, etc.; and mixtures of two or more of these excipients and/or adjuvants.

The composition may comprise a binder, such as povidone; starches; celluloses and derivatives thereof, such as microcrystalline cellulose, e.g., Avicel PH from FMC (Philadelphia, Pa.), hydroxypropyl cellulose hydroxylethyl cellulose and hydroxylpropylmethyl cellulose METHOCEL from Dow Chemical Corp. (Midland, Mich.); sucrose; dextrose; corn syrup; polysaccharides; and gelatine. The binder may be selected from the group consisting of dry binders and/or wet granulation binders. Suitable dry binders are, e.g., cellulose powder and microcrystalline cellulose, such as Avicel PH 102 and Avicel PH 200. In some embodiments the composition comprises Avicel, such as Avicel PH 102. Suitable binders for wet granulation or dry granulation are corn starch, polyvinyl pyrrolidone (povidone), vinylpyrrolidone-vinylacetate copolymer (copovidone) and cellulose derivatives like hydroxymethylcellulose, hydroxyethylcellulose, hydroxypropylcellulose and hydroxyl-propylmethylcellulose. In some embodiments the composition comprises povidone.

In some embodiments the composition comprises a filler, which may be selected from lactose, mannitol, erythritol, sucrose, sorbitol, calcium phosphate, such as calciumhydrogen phosphate, microcrystalline cellulose, powdered cellulose, confectioner's sugar, compressible sugar, dextrates, dextrin and dextrose. In some embodiments the composition comprises microcrystalline cellulose, such as Avicel PH 102 or Avicel PH 200.

In some embodiments the composition comprises a lubricant and/or a glidant. In some embodiments the composition comprises a lubricant and/or a glidant, such as talc, magnesium stearate, calcium stearate, zinc stearate, glyceryl behenate, glyceryl dibehenate, behenoyl polyoxyl-8 glycerides, polyethylene oxide polymers, sodium lauryl sulfate, magnesium lauryl sulfate, sodium oleate, sodium stearyl fumarate, stearic acid, hydrogenated vegetable oils, silicon dioxide and/or polyethylene glycol etc. In some embodiments the composition comprises magnesium stearate or glyceryl dibehenate (such as the product Compritol® 888 ATO which consists of mono-, di- and triesters of behenic acid (C22) with the diester fraction being predominant).

In some embodiments the composition comprises a disintegrant, such as sodium starch glycolate, polacrilin potassium, sodium starch glycolate, crospovidon, croscarmellose, sodium carboxymethylcellulose or dried corn starch.

The composition may comprise one or more surfactants, for example a surfactant, at least one surfactant, or two different surfactants. The term “surfactant” refers to any molecules or ions that are comprised of a water-soluble (hydrophilic) part, and a fat-soluble (lipophilic) part. The surfactant may e.g. be selected from the group consisting of anionic surfactants, cationic surfactants, nonionic surfactants, and/or zwitterionic surfactants.

As shown in the examples herein, the compositions of the invention have a very high content of the delivery agent. This very high content can be defined relative to the full content of the tablets including also the active pharmaceutical ingredient (i.e. the GLP-1 agonist) or alternatively relative to the total content of excipients excluding the active pharmaceutical ingredient. The description here below also refers to compositions consisting of specific ingredients, the GLP-1 agonist and excipients, the term consisting is to be understood to never the less encompass trace amounts of any substance with no effect on the function of the composition, which may also be referred to as consisting essential of. Such substances can be impurities remaining in preparation of the GLP-1 agonist or from the production of the salt of NAC or minimal amounts (below 1%) of any pharmaceutical acceptable excipient that do not affect the quality or absorption of the formulation.

In one embodiment the pharmaceutical composition comprises

a. a GLP-1 agonist and

b. a salt of N-(8-(2-hydroxybenzoyl)amino)caprylic acid (NAC)

wherein said salt of N-(8-(2-hydroxybenzoyl)amino)caprylic acid (NAC) constitutes at least or above 90 w/w % of the composition.

In further such embodiments the salt of NAC constitutes above 91 %, such as above 92 %, such as above 93, such as above 94 %, such as above 95 w/w % of said composition.

In further such embodiments the salt of NAC constitutes at least 91 w/w %, such as at least 92 w/w %, such as at least 93 w/w %, such as at least 94 w/w %, such as at least 95 w/w % of said composition.

In one embodiment the pharmaceutical composition comprises

a) a GLP-1 agonist and

b) a salt of N-(8-(2-hydroxybenzoyl)amino)caprylic acid (NAC),

wherein said salt of N-(8-(2-hydroxybenzoyl)amino)caprylic acid (NAC) constitutes at least 90 w/w % of the excipients of the composition.

In one embodiment the pharmaceutical composition consists of

a) a GLP-1 agonist and

b) excipients, wherein the excipients are
i. a salt of N-(8-(2-hydroxybenzoyl)amino)caprylic acid (NAC) and
ii. one or more further excipients
   wherein said salt of N-(8-(2-hydroxybenzoyl)amino)caprylic acid (NAC) constitutes at least 90 w/w % of the excipients of the composition.

In further such embodiments the salt of NAC constitutes at least at least 91 w/w %, such as at least 92 w/w %, such as at least 93 w/w %, such as at least 94 w/w %, such as at least 95 w/w % of the excipients of the composition.

In one embodiment the pharmaceutical composition comprises

a) a GLP-1 agonist and

b) a salt of N-(8-(2-hydroxybenzoyl)amino)caprylic acid (NAC),

wherein said salt of N-(8-(2-hydroxybenzoyl)amino)caprylic acid (NAC) constitutes at least 95 w/w % of the excipients of the composition.

In one embodiment the pharmaceutical composition consists of

a) a GLP-1 agonist and

b) excipients, wherein the excipients are
i. a salt of N-(8-(2-hydroxybenzoyl)amino)caprylic acid (NAC) and
ii. one or more further excipients
   wherein said salt of N-(8-(2-hydroxybenzoyl)amino)caprylic acid (NAC) constitutes at least 95 w/w % of the excipients of the composition.

In further such embodiments the salt of NAC constitutes above 95 w/w %, such as above 96 w/w %, such as above 97 w/w % or such as above 98 w/w % of the composition.

In further such embodiments the salt of NAC constitutes at least 95 w/w %, such as at least 96 w/w %, such as at least 97 w/w % or such as at least 98 w/w % of the excipients of the composition.

As mentioned above, the content of excipients, besides the delivery agent is according to the invention preferably minimal. In one embodiment, the pharmaceutical composition comprises at least one lubricant.

In one embodiment the pharmaceutical composition comprises or consists of:
a) a GLP-1 agonist,
b) a salt of N-(8-(2-hydroxybenzoyl)amino)caprylic acid (NAC) and
c) at least one lubricant.

In such embodiments the lubricant may be magnesium stearate or glyceryl dibehenate. In one embodiment the lubricant is magnesium stearate. In one embodiment the lubricant is glyceryl dibehenate.

A composition as described above wherein said salt of N-(8-(2-hydroxybenzoyl)amino)caprylic acid (NAC) constitutes at least 95 w/w % of the excipients of the composition may further be a composition wherein said salt constitutes at least or above 90 w/w % of the composition.

Likewise the compositions described above wherein said salt constitutes at least or above 90 w/w % of the composition may further be a composition wherein said salt of N-(8-(2-hydroxybenzoyl)amino)caprylic acid (NAC) constitutes at least 95 w/w % of the excipients of the composition.

The pharmaceutical composition may further be a composition wherein the salt of N-(8-(2-hydroxybenzoyl)amino)caprylic acid (NAC) is selected from the group consisting of the sodium salt, potassium salt and/or calcium salt of of N-(8-(2-hydroxybenzoyl)amino)caprylic acid (NAC) or alternatively from the group consisting of just the sodium salt and the potassium salt. In one embodiment the salt of N-(8-(2-hydroxybenzoyl)amino)caprylic acid (NAC) is sodium N-(8-(2-hydroxybenzoyl)amino)caprylate.

In embodiments wherein said salt of N-(8-(2-hydroxybenzoyl)amino)caprylic acid (NAC) constitutes at least 90 w/w % of the excipients of the composition, the composition comprises at most 10 w/w % of any further excipients, such as binder, filler, and/or lubricant/glidant. In some embodiments the composition comprises at least or above 90 w/w % delivery agent, and less than 5 w/w % of any further excipients, such as binder, filler, and/or lubricant/glidant. In one embodiment the pharmaceutical composition comprises at least 90 w/w % delivery agent and less than 5 w/w % lubricant. In one embodiment the pharmaceutical composition comprises at least 90 w/w % delivery agent and less than 3 w/w % lubricant.

In some embodiments the composition comprises at least or above 90 w/w % delivery agent and 0.1-5 w/w %, such as 0.5-4 w/w % or 1-3 w/w %, of lubricant. In further such embodiments the composition comprises 2-2.5 w/w % of lubricant.

In embodiments wherein said salt of N-(8-(2-hydroxybenzoyl)amino)caprylic acid (NAC) constitutes at least 95 w/w % of the excipients of the composition, the composition comprises at most 5 w/w % of any further excipients, such as binder, filler, and/or lubricant/glidant. In some embodiments the composition comprises at least 95 w/w % delivery agent and less than 5 w/w % lubricant. In one embodiment the pharmaceutical composition comprises at least 95 w/w % delivery agent and less than 3 w/w % lubricant.

In some embodiments the composition comprises at least 95 w/w % delivery agent and 0.1-5 w/w %, such as 0.5-4 w/w % or 1-3 w/w %, of lubricant. In further such embodiments the composition comprises 2-2.5 w/w % of lubricant.

The pharmaceutical composition according to the invention is preferably produced in a dosage form suitable for oral administration as described herein below. In the following the absolute amounts of the ingredients of the composition of the invention are provided with reference to the content in a dosage unit i.e. per tablet, capsule or sachet.

The pharmaceutical compositions of the invention may in a further embodiment comprise at most 1000 mg of said salt of N-(8-(2-hydroxybenzoyl)amino)caprylic acid per dose unit. In one embodiment the invention relates to a composition wherein a dose unit comprises at most 500 mg of said salt.

In some embodiments the amount of the salt of N-(8-(2-hydroxybenzoyl) amino)caprylic acid per dose unit is at least 0.05 mmol, such as at least 0.075 mmol, such as at least 0.1 mmol, such as at least 0.125 mmol, such as at least 0.15 mmol, such as at least 0.20 mmol, at least 0.25 mmol, at least 0.30 mmol, at least 0.35 mmol, at least 0.40 mmol, at least 0.45 mmol, at least 0.50 mmol, at least 0.55 mmol or at least 0.60 mmol.

In some embodiments the amount of the salt of N-(8-(2-hydroxybenzoyl)amino)caprylic acid per dosage unit of the composition is up to 3 mmol, such as up to 2.75 mmol, such as up to 2.5 mmol, such as up to 2.25 mmol, such as 2 mmol, such as up to 1.5 mmol, up to 1 mmol, up to 0.75 mmol, up to 0.6 mmol, up to 0.5 mmol, up to 0.4 mmol, up to 0.3 mmol and up to 0.2 mmol.

In some embodiments the amount of the salt of N-(8-(2-hydroxybenzoyl) amino)caprylic acid per dose unit of the composition is in the range of 0.05-3 mmol, 0.10-2.5 mmol, 0.15-2.0 mmol, 0.20-1.5 mmol, 0.25-1.0 mmol, 0.30-0.75 mmol or such as 0.45-0.65 mmol.

In some embodiments, where the salt of NAC is SNAC, the amount of SNAC in the composition is at least 20 mg, such as at least 25 mg, such as at least 50 mg, such as at least 75 mg, at least 100 mg, at least 125 mg, at least 150 mg, at least 175 mg, at least 200 mg, at least 225 mg, at least 250 mg, at least 275 mg and at least 300 mg per dose unit.

In some embodiments, where the salt of NAC is SNAC, the amount of SNAC in the composition is up to 800 mg, such as up to 600 mg, such as up to 575 mg, such as up to 550 mg, up to 525 mg, up to 500 mg, up to 475 mg, up to 450 mg, up to 425 mg, up to 400 mg, up to 375 mg, up to 350 mg, up to 325 mg per dose unit, or up to 300 mg per dose unit.

In some embodiments, where the salt of NAC is SNAC, the amount of SNAC in the composition is in the range of 20-800 mg, such as 25-600 mg, such as 50-500 mg, such as 50-400 mg, such as 75-400 mg, such as 80-350 mg or such as from around 100 to around 300 mg per dose unit.

In one embodiment, where the salt of NAC is SNAC, the amount of SNAC is in the range of 20-200 mg, such as 25-175 mg, such as 75-150 mg, such as 80-120 mg such as around 100 mg per dose unit.

In one embodiment, where the salt of NAC is SNAC, the amount of SNAC is in the range of 200-800 mg, such as 250-400 mg, such as 250-350 mg, such as 275-325 mg, such as around 300 mg per dose unit.

In an embodiment, a dose unit of the pharmaceutical compositions of the invention comprises 0.1-100 mg or 0.2 to 100 mg of the GLP-1 agonist.

In some embodiments a dose unit of the composition comprises an amount of GLP-1 agonist is in the range of 0.2 to 50 mg or 1 to 40 mg.

In some embodiments a dose unit comprises 0.5-5 mg of the GLP-1 agonist, such as 0.75-4.5 mg, such as 1, 1.5, 2, 2.5 or 3 mg or 3.5, 4, 4.5 mg, such as 1-3 or 3-5 mg of the GLP-1 agonist per dose unit.

In some embodiments a dose unit comprises 2 to 20 mg of the GLP-1 agonist, such as 2-15 mg, such as 2, 3, 4, 5, 6 or 7 mg, such as 2, 3, 4 or 5 mg, or such as 8, 10, 12 or 14 mg, such as 15 mg or such as 20 mg of the GLP-1 agonist per dose unit.

In some embodiments a dose unit comprises 5 to 50 mg of the GLP-1 agonist, such as 10-45 mg, such as 20, 30 or 40 mg, or such as 25, 35, or 45 mg, or such as 30-50 mg or such as 20-40 mg of the GLP-1 agonist per dose unit.

The amount of GLP-1 agonist may be varied depending on identity of the GLP-1 agonist and the effect desired, i.e. a higher content may be relevant for treating obesity compared to diabetes.

In a preferred embodiment a unit dose of the composition comprises 0.5-25 mg magnesium stearate, such as 1-10 mg, such as 2-8 mg or such as 2-5 mg magnesium stearate.

In a preferred embodiment the amount of magnesium stearate is determined relative to the amount of the salt of NAC, such as SNAC, such that a unit dose of the composition comprises 1-8 mg magnesium stearate or such as 2-5 mg magnesium stearate or 2-3 mg magnesium stearate per 100 mg salt of N-(8-(2-hydroxybenzoyl)amino)caprylic acid, such as SNAC.

In a preferred embodiment a unit dose of the composition comprises 80-120 mg SNAC, 0.5-5 mg GLP-1 agonist and 2-3 mg lubricant.

In a preferred embodiment a unit dose of the composition comprises 80-120 mg SNAC, 1.5-10 mg GLP-1 agonist and 2-3 mg lubricant.

In a preferred embodiment a unit dose of the composition comprises 80-120 mg SNAC, 5-50 mg GLP-1 agonist and 2-3 mg lubricant.

In a preferred embodiment a unit dose of the composition comprises 250-350 mg SNAC, 0.5-5 mg GLP-1 agonist and 3-10 mg lubricant.

In a preferred embodiment a unit dose of the composition comprises 250-350 mg SNAC, 1.5-10 mg GLP-1 agonist and 3-10 mg lubricant.

In a preferred embodiment a unit dose of the composition comprises 250-350 mg SNAC, 5-50 mg GLP-1 agonist and 3-10 mg lubricant.

In one embodiment the pharmaceutical composition of the invention has a fast release in vitro. Release or dissolution may be tested as known in the art and as described here in Assay I. The release may be expressed as the amount of the GLP-1 agonist measured in solution after a given period relative to the total content of the GLP-1 agonist of the composition. The relative amount may be given in percentage. In one embodiment the release of the GLP-1 agonist from the pharmaceutical composition of the invention is at least 85% within 15 minutes or at least 95% within 30 minutes. In one such embodiment the release is measured at pH 6.8.

In one embodiment the pharmaceutical composition comprises
a) a GLP-1 agonist and
b) a salt of N-(8-(2-hydroxybenzoyl)amino)caprylic acid, wherein the release of the GLP-1 agonist reaches 85% within 15 minutes or 95% within 30 minutes. In one embodiment the release is measured at pH 6.8.

In one embodiment the pharmaceutical composition of the invention provides an early exposure in vivo. In one embodiment the pharmaceutical composition of the invention provides an increased exposure in vivo. In one embodiment the pharmaceutical composition of the invention provides an increased early exposure in vivo. Such in vivo exposure may be tested in a relevant model, such as the Assay III described herein. As seen in table 3.1 the compositions disclosed herein demonstrate an increased early dose corrected exposure in beagle dogs within the first 30 minutes after oral dosing. The exposure may also be measured over a predetermined time period and the accumulative dose corrected exposure (AUC) calculated, such as for t=0-30 minutes as also provided in table 3.1 for the compositions described herein.

In one embodiment the invention relates to a pharmaceutical composition wherein the dose corrected exposure at t=30 min is increased relative to the composition described in WO2013/139694, which comprise the additional excipients microcrystalline cellulose and povidone. The Reference composition for a given GLP-1 agonist should preferable be prepared with two granules as disclosed for type F and H in WO2013/139694, and prepared by substituting semaglutide/Compound A with the GLP-1 agonist of interest.

In one embodiment the pharmaceutical composition comprises
a) a GLP-1 agonist and
b) a salt of N-(8-(2-hydroxybenzoyl)amino)caprylic acid,
   wherein the dose corrected exposure at t=30 min is increased relative to a composition of type F/H of WO2013/139694.

In one embodiment the pharmaceutical composition comprises
a) a GLP-1 agonist and
b) a salt of N-(8-(2-hydroxybenzoyl)amino)caprylic acid, wherein the dose corrected exposure (AUC) for t=0-30 min is increased relative to a composition of type F/H of WO2013/139694.

In one embodiment the dose corrected exposure (AUC) for t=0-30 min is increased at least 1.5 fold, such as 2 fold compared to a composition of type F/H of WO2013/139694.

### Dosage Form

The composition may be administered in several dosage forms, for example as a tablet; a coated tablet; a sachet or a capsule such as hard or soft shell capsules.

The composition may further be compounded in a drug carrier or drug delivery system, e.g. in order to improve stability and/or solubility or further improve bioavailability.

The composition may be a freeze-dried or spray-dried composition.

The composition may be in the form of a dose unit, such as a tablet. In some embodiments the weight of the unit dose is in the range of 50 mg to 1000 mg, such as in the range of 50-750 mg, or such as in the range of 100-500 mg.

In some embodiments the weight of the dose unit is in the range of 75 mg to 350 mg, such as in the range of 100-300 mg or such as in the range of 200-350 mg.

In some embodiments the weight of the dose unit is in the range of 100 mg to 400 mg, such as in the range of 50-300 mg or such as in the range of 200-400 mg.

In some embodiments the composition may be granulated prior to being compacted. The composition may comprise an intragranular part and/or an extragranular part, wherein the intragranular part has been granulated and the extragranular part has been added after granulation.

The intragranular part may comprise the GLP-1 agonist, the delivery agent and/or an excipient, such as a lubricant and/or glidant. In some embodiments the intragranular part comprises the delivery agent and a lubricant and/or a glidant.

The extragranular part may comprise a GLP-1 agonist, and/or a lubricant and/or a glidant, such as magnesium stearate. In some embodiments the extragranular part comprises the GLP-1 agonist. In some embodiments the extragranular part comprises an excipient, such as a lubricant and/or glidant, such as magnesium stearate.

In further embodiments the intragranular part comprises the GLP-1 agonist, the delivery agent and the lubricant and/or a glidant. In such embodiments the granulate may be directly compressed into tablets and the tablets have no extragranular part.

### Preparation of Composition

Preparation of a composition according to the invention may be performed according to methods known in the art.

To prepare a dry blend of tabletting material, the various components are weighed, optionally delumped or sieved and then combined. The mixing of the components may be carried out until a homogeneous blend is obtained.

If granules are to be used in the tabletting material, granules may be produced in a manner known to a person skilled in the art, for example using wet granulation methods known for the production of “built-up” granules or “broken-down” granules. Methods for the formation of built-up granules may operate continuously and comprise, for example simultaneously spraying the granulation mass with granulation solution and drying, for example in a drum granulator, in pan granulators, on disc granulators, in a fluidized bed, by spray-drying, spray-granulation or spray-solidifying, or operate discontinuously, for example in a fluidized bed, in a rotary fluid bed, in a batch mixer, such as a high shear mixer or a low shear mixer, or in a spray-drying drum. Methods for the production of broken-down granules, which may be carried out discontinuously and in which the granulation mass first forms a wet aggregate with the granulation solution, which is subsequently comminuted or by other means formed into granules of the desired size and the granules may then be dried. Suitable equipment for the wet granulation step are planetary mixers, low shear mixers, high shear mixers, extruders and spheronizers, such as an apparatus from the companies Loedige, Glatt, Diosna, Fielder, Collette, Aeschbach, Alexanderwerk, Ytron, Wyss & Probst, Werner & Pfleiderer, HKD, Loser, Fuji, Nica, Caleva and Gabler. Granules may also be formed by dry granulation techniques in which one or more of the excipient(s) and/or the active pharmaceutical ingredient is compressed to form relatively large moldings, for example slugs or ribbons, which are comminuted by grinding, and the ground material serves as the tabletting material to be later compacted. Suitable equipment for dry granulation is, but not limited to, roller compaction equipment from Gerteis such as Gerteis MICRO-PACTOR, MINI-PACTOR and MACRO-PACTOR.

The terms “granulate” and “granules” are used interchangeably herein to refer to particles of composition material which may be prepared as described above.

To compact the tabletting material into a solid oral dosage form, for example a tablet, a tablet press may be used. In a tablet press, the tabletting material is filled (e.g. force fed or gravity fed) into a die cavity. The tabletting material is then compacted by a set of punches applying pressure. Subsequently, the resulting compact, or tablet is ejected from the tablet press. The above mentioned tabletting process is subsequently referred to herein as the “compaction process”. Suitable tablet presses include, but are not limited to, rotary tablet presses and eccentric tablet presses. Examples of tablet presses include, but are not limited to, the Fette 102i (Fette GmbH), the Korsch XL100, the Korsch PH 106 rotary tablet press (Korsch AG, Germany), the Korsch EK-O eccentric tabletting press (Korsch AG, Germany) and the Manesty F-Press (Manesty Machines Ltd., United Kingdom).

In one embodiment the composition comprises at least one granulate. In one embodiment the composition comprises one type of granulate. The composition may alternatively comprise two types of granulates.

In some embodiments the method of preparation of the tablet comprises; a) granulating a mixture comprising the delivery agent and optionally a lubricant; b) blending the granulate of a) with a GLP-1 agonist and optionally additional lubricant, and then c) compressing the blend of b) into tablets.

In some embodiments the method of preparation of the tablet comprises; a) granulating a mixture comprising the delivery agent, the GLP-1 agonist and optionally a lubricant and b) compressing the granulate of a) into tablets and optionally including additional lubricant.

In general, granulates may be prepared by wet, melt or dry granulation, preferably dry granulation.

### Pharmaceutical Indications

The present invention also relates to a composition of the invention for use as a medicament. In particular embodiments the composition of the invention may be used for the following medical treatments, all preferably relating one way or the other to diabetes and/or obesity:

(i) prevention and/or treatment of all forms of diabetes, such as hyperglycemia, type 2 diabetes, impaired glucose tolerance, type 1 diabetes, non-insulin dependent diabetes, MODY (maturity onset diabetes of the young), gestational diabetes, and/or for reduction of HbA1C;

(ii) delaying or preventing diabetic disease progression, such as progression in type 2 diabetes, delaying the progression of impaired glucose tolerance (IGT) to insulin requiring type 2 diabetes, and/or delaying the progression of non-insulin requiring type 2 diabetes to insulin requiring type 2 diabetes;

(iii) improving β-cell function, such as decreasing β-cell apoptosis, increasing β-cell function and/or β-cell mass, and/or for restoring glucose sensitivity to β-cells;

(iv) prevention and/or treatment of cognitive disorders;

(v) prevention and/or treatment of eating disorders, such as obesity, e.g. by decreasing food intake, reducing body weight, suppressing appetite, inducing satiety; treating or preventing binge eating disorder, bulimia nervosa, and/or obesity induced by administration of an antipsychotic or a steroid; reduction of gastric motility; and/or delaying gastric emptying;

(vi) prevention and/or treatment of diabetic complications, such as neuropathy, including peripheral neuropathy; nephropathy; or retinopathy;

(vii) improving lipid parameters, such as prevention and/or treatment of dyslipidemia, lowering total serum lipids; lowering HDL; lowering small, dense LDL; lowering VLDL: lowering triglycerides; lowering cholesterol; increasing HDL; lowering plasma levels of lipoprotein a (Lp(a)) in a human; inhibiting generation of apolipoprotein a (apo(a)) in vitro and/or in vivo;

(iix) prevention and/or treatment of cardiovascular diseases, such as syndrome X; atherosclerosis; myocardial infarction; coronary heart disease; stroke, cerebral ischemia; an early cardiac or early cardiovascular disease, such as left ventricular hypertrophy; coronary artery disease; essential hypertension; acute hypertensive emergency; cardiomyopathy; heart insufficiency; exercise tolerance; chronic heart failure; arrhythmia; cardiac dysrhythmia; syncopy; atheroschlerosis; mild chronic heart failure; angina pectoris; cardiac bypass reocclusion; intermittent claudication (atheroschlerosis oblitterens); diastolic dysfunction; and/or systolic dysfunction;

(ix) prevention and/or treatment of gastrointestinal diseases, such as inflammatory bowel syndrome; small bowel syndrome, or Crohn's disease; dyspepsia; and/or gastric ulcers;

(x) prevention and/or treatment of critical illness, such as treatment of a critically ill patient, a critical illness poly-nephropathy (CIPNP) patient, and/or a potential CIPNP patient; prevention of critical illness or development of CIPNP; prevention, treatment and/or cure of systemic inflammatory response syndrome (SIRS) in a patient; and/or for the prevention or reduction of the likelihood of a patient suffering from bacteraemia, septicaemia, and/or septic shock during hospitalisation; and/or

(xi) prevention and/or treatment of polycystic ovary syndrome (PCOS).

In a particular embodiment, the indication is selected from the group consisting of (i)-(iii) and (v)-(iix), such as indications (i), (ii), and/or (iii); or indication (v), indication (vi), indication (vii), and/or indication (iix). In another particular embodiment, the indication is (i). In a further particular embodiment the indication is (v). In a still further particular embodiment the indication is (iix). In some embodiments the indications are type 2 diabetes and/or obesity.

### Method of Treatment

The invention further relates to a method of treating a subject in need thereof, comprising administering a therapeutically effective amount of a composition according to the present invention to said subject. In one embodiment the method of treatment is for treatment of diabetes or obesity and/or the further indications specified above.

In some embodiments, a method for treating diabetes is described comprising administering to a subject in need thereof a therapeutically effective amount of a pharmaceutical composition comprising a GLP-1 agonist, a salt of N-(8-(2-hydroxybenzoyl)amino)caprylic acid (NAC), and optionally, a lubricant.

In some embodiments, a method for treating diabetes is described comprising administering to a subject in need thereof a therapeutically effective amount of a pharmaceutical composition comprising 0.5-50 mg of a GLP-1 agonist, 50-500 mg of salt of N-(8-(2-hydroxybenzoyl)amino)caprylic acid (NAC), and 1-10 mg lubricant. In a preferred embodiment, the salt of N-(8-(2-hydroxybenzoyl)amino)caprylic acid constitutes at least 90% (w/w) of the composition.

In a preferred embodiment, the salt of N-(8-(2-hydroxybenzoyl)amino)caprylic acid constitutes at least 95% (w/w) of the excipients of the composition.

In an alternative embodiment, a method for treating diabetes is described comprising administering to a subject in need thereof a therapeutically effective amount of a pharmaceutical composition comprising about 1-14 mg of a GLP-1 agonist, about 100-300 mg of salt of N-(8-(2-hydroxybenzoyl)amino)caprylic acid (NAC), and about 2-8 mg of magnesium stearate.

In some embodiments, the GLP-1 agonist is semaglutide having a formula of N-epsilon26-[2-(2-{2-[2-(2-{2-[(S)-4-carboxy-4-(17-carboxy- heptadecanoylamino)butyrylamino]ethoxy}ethoxy)acetylamino]ethoxy}ethoxy)acetyl] [Aib8,Arg34]GLP-1(7-37) and the salt of N-(8-(2-hydroxybenzoyl)amino)caprylic acid (NAC) is sodium N-(8-(2-hydroxybenzoyl)amino)caprylic acid (SNAC). Various examples of a lubricant are described, including magnesium stearate. The composition is administered orally and is in a form of a table, capsule or a sachet.

In a further such embodiments one or more dose units may be administered to said subject in need.

### Combination Treatment

The treatment with a composition according to the present invention may also be combined with one or more additional active pharmaceutical ingredient(s), e.g. selected from antidiabetic agents, antiobesity agents, appetite regulating agents, antihypertensive agents, agents for the treatment and/or prevention of complications resulting from or associated with diabetes and agents for the treatment and/or prevention of complications and disorders resulting from or associated with obesity. Examples of these pharmacologically active substances are: Insulin, sulphonylureas, biguanides, meglitinides, glucosidase inhibitors, glucagon antagonists, DPP-IV (dipeptidyl peptidase-IV) inhibitors, sodium glucose linked transporter 2 (SGLT2) inhibitors; canagliflozin, dapagliflozin, empagliflozin, ertugliflozin, ipragliflozin, tofogliflozin, luseogliflozin, bexagliflozin, remogliflozin etabonate and sotagliflozin, particulally dapagliflozin and empagliflozin, inhibitors of hepatic enzymes involved in stimulation of gluconeogenesis and/or glycogenolysis, glucose uptake modulators, compounds modifying the lipid metabolism such as antihyperlipidemic agents as HMG CoA inhibitors (statins), Gastric Inhibitory Polypeptides (GIP analogues), compounds lowering food intake, RXR agonists and agents acting on the ATP-dependent potassium channel of the β-cells; Cholestyramine, colestipol, clofibrate, gemfibrozil, lovastatin, pravastatin, simvastatin, probucol, dextrothyroxine, neteglinide, repaglinide; β-blockers such as alprenolol, atenolol, timolol, pindolol, propranolol and metoprolol, ACE (angiotensin converting enzyme) inhibitors such as benazepril, captopril, enalapril, fosinopril, lisinopril, alatriopril, quinapril and ramipril, calcium channel blockers such as nifedipine, felodipine, nicardipine, isradipine, nimodipine, diltiazem and verapamil, and α-blockers such as doxazosin, urapidil, prazosin and terazosin; CART (cocaine amphetamine regulated transcript) agonists, NPY (neuropeptide Y) antagonists, PYY agonists, Y2 receptor agonists, Y4 receptor agonists, mixed Y2/Y4 receptor agonists, MC4 (melanocortin 4) agonists, orexin antagonists, TNF (tumour necrosis factor) agonists, CRF (corticotropin releasing factor) agonists, CRF BP (corticotropin releasing factor binding protein) antagonists, urocortin agonists, β3 agonists, oxyntomodulin and analogues, MSH (melanocyte-stimulating hormone) agonists, MCH (melanocyte-concentrating hormone) antagonists, CCK (cholecystokinin) agonists, serotonin re-uptake inhibitors, serotonin and noradrenaline re-uptake inhibitors, mixed serotonin and noradrenergic compounds, 5HT (serotonin) agonists, bombesin agonists, galanin antagonists, growth hormone, growth hormone releasing compounds, TRH (thyreotropin releasing hormone) agonists, UCP 2 or 3 (uncoupling protein 2 or 3) modulators, leptin agonists, DA agonists (bromocriptin, doprexin), lipase/amylase inhibitors, RXR (retinoid X receptor) modulators, TR β agonists; histamine H3 antagonists, Gastric Inhibitory Polypeptide agonists or antagonists (GIP analogues), gastrin and gastrin analogues.

The invention as described herein is, without limitation hereto, further defined by the embodiments described here below and the claims of the document.

### EMBODIMENTS

1. A pharmaceutical composition comprising
a) a GLP-1 agonist and
b) a salt of N-(8-(2-hydroxybenzoyl)amino)caprylic acid

wherein said salt of N-(8-(2-hydroxybenzoyl)amino)caprylic acid constitutes at least 90 w/w % of the composition.

2. A pharmaceutical composition comprising
a) a GLP-1 agonist and
b) a salt of N-(8-(2-hydroxybenzoyl)amino)caprylic acid

wherein said salt of N-(8-(2-hydroxybenzoyl)amino)caprylic acid constitutes at least 90 w/w %, such as at least 95 w/w % of the excipients of the composition.

3. A pharmaceutical composition consisting of
a) a GLP-1 agonist and
b) excipients, wherein the excipients are
   i. a salt of N-(8-(2-hydroxybenzoyl)amino)caprylic acid and
   ii. one or more further excipients

wherein said salt of N-(8-(2-hydroxybenzoyl)amino)caprylic acid constitutes at least 90 w/w %, such as at least 95 w/w % of the excipients of the composition.

4. The pharmaceutical composition according to any of the previous embodiments 1-3, wherein the composition comprises at least one lubricant.

5. A pharmaceutical composition consisting of:
a) a GLP-1 agonist,
b) a salt of N-(8-(2-hydroxybenzoyl)amino)caprylic acid and
c) at least one lubricant 6. The pharmaceutical composition according to any of the previous embodiments 4 and 5, wherein the lubricant is magnesium stearate.

7. The pharmaceutical composition according any of the previous embodiments, wherein the composition comprises 1-8 mg, such as 2-5 mg or such as 2-3 mg magnesium stearate per 100 mg salt of N-(8-(2-hydroxybenzoyl)amino)caprylic acid.

8. The pharmaceutical composition according to any of the previous embodiments 2-7, wherein said salt of N-(8-(2-hydroxybenzoyl)amino)caprylic acid constitutes at least 90 w/w % of the composition.

9. The pharmaceutical composition according to any of the previous embodiments 1, 5-8, wherein said salt of N-(8-(2-hydroxybenzoyl)amino)caprylic acid constitutes at least 95 w/w % of the excipients of the composition.

10. The pharmaceutical composition according to any of the previous embodiments, wherein the salt of N-(8-(2-hydroxybenzoyl)amino)caprylic acid is selected from the group consisting of the sodium salt, potassium salt and/or calcium salt of of N-(8-(2-hydroxybenzoyl)amino)caprylic acid.

11. The pharmaceutical composition according to any of the previous embodiments, wherein the salt of N-(8-(2-hydroxybenzoyl)amino)caprylic acid is sodium N-(8-(2-hydroxybenzoyl)amino)caprylate (SNAC).

12. The pharmaceutical composition according to any of the previous embodiments, wherein a dose unit comprises at most 1000 mg of said salt of N-(8-(2-hydroxybenzoyl)amino)caprylic acid.

13. The pharmaceutical composition according to any of the previous embodiments, wherein a dose unit comprises 0.5-50 mg of the GLP-1 agonist.

14. The pharmaceutical composition according to any of the previous embodiments, wherein the GLP-1 agonist has T ½ of at least 24 hours in minpigs.

15. The pharmaceutical composition according to any of the previous embodiments, wherein the GLP-1 agonist has an EC50 (without HSA) of at most 100 pM, such as at most 50.

16. The pharmaceutical composition according to any of the previous embodiments, wherein the GLP-1 agonist is selected from the group consisting of: liraglutide, semaglutide, GLP-1 agonist B and GLP-1 agonist C.

17. The pharmaceutical composition according to any of the previous embodiments, wherein the GLP-1 agonist is selected from the group consisting of: semaglutide and GLP-1 agonist C.

18. The pharmaceutical composition according to any of the previous embodiments, wherein the composition comprises at least one granulate.

19. The pharmaceutical composition according to previous embodiment 18, wherein the at least one granulate comprises the salt of N-(8-(2-hydroxybenzoyl)amino)caprylic acid.

20. The pharmaceutical composition according to any of the previous embodiments 18-19, wherein the at least one granulate further comprises a lubricant, such as magnesium stearate.

21. The pharmaceutical composition according to any of the previous embodiments 18-20, wherein the at least one granulate further comprises the GLP-1 agonist.

22. The pharmaceutical composition according to any of the previous embodiment 18-21, wherein the at least one granulate is prepared by dry granulation, such as by roller compaction.

23. The pharmaceutical composition according to any of the previous embodiment 18-22, wherein the composition comprises an extragranular part.

24. The pharmaceutical composition according to any of the previous embodiment 18-23, wherein the extragranular part of the composition comprises a lubricant or glidant, such as magnesium stearate and/or the GLP-1 agonist.

25. A pharmaceutical composition comprising
a) 0.5-50 mg of a GLP-1 agonist and
b) 20-800 mg, such as 25-600, such as 50-500 mg of a salt of N-(8-(2-hydroxybenzoyl)amino)caprylic acid,

wherein said salt of N-(8-(2-hydroxybenzoyl)amino)caprylic acid constitutes at least 90 w/w %, such as at least 95 w/w % of the excipients of the composition and wherein the GLP-1 agonist is semaglutide.

26. A pharmaceutical composition comprising
a) 1-25 mg of a GLP-1 agonist and
b) 50-400 mg of a salt of N-(8-(2-hydroxybenzoyl)amino)caprylic acid, wherein said salt of N-(8-(2-hydroxybenzoyl)amino)caprylic acid constitutes at least 90 w/w %, such as at least 95 w/w % of the excipients of the composition and wherein the GLP-1 agonist is semaglutide.

27. A pharmaceutical composition comprising
a) 1-15 mg of a GLP-1 agonist and
b) 75-150 mg of a salt of N-(8-(2-hydroxybenzoyl)amino)caprylic acid, wherein said salt of N-(8-(2-hydroxybenzoyl)amino)caprylic acid constitutes at least 90 w/w %, such as at least 95 w/w % of the excipients of the composition and wherein the GLP-1 agonist is semaglutide.

28. A pharmaceutical composition comprising
a) 1-15 mg of a GLP-1 agonist and
b) 75-125 mg of a salt of N-(8-(2-hydroxybenzoyl)amino)caprylic acid, wherein said salt of N-(8-(2-hydroxybenzoyl)amino)caprylic acid constitutes at least 90 w/w %, such as at least 95 w/w % of the excipients of the composition and wherein the GLP-1 agonist is semaglutide.

29. A pharmaceutical composition comprising
a) 1-15 mg of a GLP-1 agonist and
b) 80-120 mg of a salt of N-(8-(2-hydroxybenzoyl)amino)caprylic acid, wherein said salt of N-(8-(2-hydroxybenzoyl)amino)caprylic acid constitutes at least 90 w/w %, such as at least 95 w/w % of the excipients of the composition and wherein the GLP-1 agonist is semaglutide.

30. A pharmaceutical composition comprising
a) 1-15 mg of a GLP-1 agonist and
b) 200-400 mg of a salt of N-(8-(2-hydroxybenzoyl)amino)caprylic acid,

wherein said salt of N-(8-(2-hydroxybenzoyl)amino)caprylic acid constitutes at least 90 w/w %, such as at least 95 w/w % of the excipients of the composition and wherein the GLP-1 agonist is semaglutide.

31. A pharmaceutical composition comprising
a) 1-15 mg of a GLP-1 agonist and
b) 250-350 mg of a salt of N-(8-(2-hydroxybenzoyl)amino)caprylic acid, wherein said salt of N-(8-(2-hydroxybenzoyl)amino)caprylic acid constitutes at least 90 w/w %, such as at least 95 w/w % of the excipients of the composition and wherein the GLP-1 agonist is semaglutide.

32. A pharmaceutical composition comprising
a) 0.1-25 mg of a GLP-1 agonist and
b) 20-800 mg, such as 25-600, 50-500 mg of a salt of N-(8-(2-hydroxybenzoyl)amino)caprylic acid,

wherein said salt of N-(8-(2-hydroxybenzoyl)amino)caprylic acid constitutes at least 90 w/w %, such as at least 95 w/w % of the excipients of the composition and wherein the GLP-1 agonist is GLP-1 agonist C.

33. A pharmaceutical composition comprising
a) 1-25 mg of a GLP-1 agonist and
b) 50-400 mg of a salt of N-(8-(2-hydroxybenzoyl)amino)caprylic acid, wherein said salt of N-(8-(2-hydroxybenzoyl)amino)caprylic acid constitutes at least 90 w/w %, such as at least 95 w/w % of the excipients of the composition and wherein the GLP-1 agonist is GLP-1 agonist C.

34. A pharmaceutical composition comprising
a) 1-15 mg of a GLP-1 agonist and
b) 75-150 mg of a salt of N-(8-(2-hydroxybenzoyl)amino)caprylic acid, wherein said salt of N-(8-(2-hydroxybenzoyl)amino)caprylic acid constitutes at least 90 w/w %, such as at least 95 w/w % of the excipients of the composition and wherein the GLP-1 agonist is GLP-1 agonist C.

35. A pharmaceutical composition comprising
c) 1-15 mg of a GLP-1 agonist and
d) 75-125 mg of a salt of N-(8-(2-hydroxybenzoyl)amino)caprylic acid, wherein said salt of N-(8-(2-hydroxybenzoyl)amino)caprylic acid constitutes at least 90 w/w %, such as at least 95 w/w % of the excipients of the composition and wherein the GLP-1 agonist is GLP-1 agonist C.

36. A pharmaceutical composition comprising
c) 1-15 mg of a GLP-1 agonist and
d) 80-120 mg of a salt of N-(8-(2-hydroxybenzoyl)amino)caprylic acid, wherein said salt of N-(8-(2-hydroxybenzoyl)amino)caprylic acid constitutes at least 90 w/w %, such as at least 95 w/w % of the excipients of the composition and wherein the GLP-1 agonist is GLP-1 agonist C.

37. A pharmaceutical composition comprising
a) 1-15 mg of a GLP-1 agonist and
b) 200-400 mg of a salt of N-(8-(2-hydroxybenzoyl)amino)caprylic acid, wherein said salt of N-(8-(2-hydroxybenzoyl)amino)caprylic acid constitutes at least 90 w/w %, such as at least 95 w/w % of the excipients of the composition and wherein the GLP-1 agonist is GLP-1 agonist C.

38. A pharmaceutical composition comprising
a) 1-15 mg of a GLP-1 agonist and
b) 250-350 mg of a salt of N-(8-(2-hydroxybenzoyl)amino)caprylic acid, wherein said salt of N-(8-(2-hydroxybenzoyl)amino)caprylic acid constitutes at least 95 w/w % of the excipients of the composition and wherein the GLP-1 agonist is GLP-1 agonist C.

39. The pharmaceutical composition according to any of the embodiments 25-38, further comprising 1-10 mg lubricant, such as magnesium stearate.

40. The pharmaceutical composition according to any of the embodiments 25-38, further comprising 1-8 mg, such as 2-5 mg or such as 2-3 mg magnesium stearate per 100 mg salt of N-(8-(2-hydroxybenzoyl)amino)caprylic acid.

41. The pharmaceutical composition according to any of the previous embodiment, wherein the composition is for oral administration.

42. The pharmaceutical composition according to any of the previous embodiments, wherein the composition is a solid composition.

43. The pharmaceutical composition according to the previous embodiments, wherein the composition is a solid composition, such as a tablet, a capsule or a sachet.

44. A pharmaceutical composition comprising
a) a GLP-1 agonist and
b) a salt of N-(8-(2-hydroxybenzoyl)amino)caprylic acid, wherein the release of the GLP-1 agonist reaches 85% within 15 minutes or 95% within minutes.

45. A pharmaceutical composition comprising
a) a GLP-1 agonist and
b) a salt of N-(8-(2-hydroxybenzoyl)amino)caprylic acid,

wherein the dose corrected exposure at t=30 min is increased relative to a reference composition of type F/H of WO2013/139694.

46. A pharmaceutical composition comprising
a) a GLP-1 agonist and
b) a salt of N-(8-(2-hydroxybenzoyl)amino)caprylic acid,

wherein the dose corrected exposure (AUC) for t=0-30 min is increased relative to a reference composition of type F/H of WO2013/139694.

47. The pharmaceutical composition according to previous embodiment 46, wherein the dose corrected exposure (AUC) for T=0-30 min is increased at least 1.5 fold, such as at least 2 fold.

48. The pharmaceutical composition according to any of the embodiments 25-40 further defined by the features of one or more of the embodiments 8, 10, 11, 18-24 and 41-47.

49. The pharmaceutical composition according to embodiments 44, 45, 46 or 47 further defined by the features of one or more of the embodiments 4, 6-24 and 41-43.

50. A pharmaceutical composition according to any of the previous embodiments for use in medicine.

51. A pharmaceutical composition according to any of the previous embodiments for use in a method of treatment of diabetes and/or obesity.

52. A method of treatment of a subject in need thereof, wherein the method comprises administering a therapeutically active amount of a composition according to any of the previous embodiments to said subject.

### Further Embodiments

1. A pharmaceutical composition comprising
a) a GLP-1 agonist and
b) a salt of N-(8-(2-hydroxybenzoyl)amino)caprylic acid,

wherein said salt of N-(8-(2-hydroxybenzoyl)amino)caprylic acid constitutes at least 95 w/w % of the excipients of the composition.

2. The pharmaceutical composition according to embodiment 1, further comprising at least one lubricant.

3. A pharmaceutical composition consisting of:
a) a GLP-1 agonist,
b) a salt of N-(8-(2-hydroxybenzoyl)amino)caprylic acid and
c) at least one lubricant.

4. The pharmaceutical composition according to any of the previous embodiments, wherein said salt of N-(8-(2-hydroxybenzoyl)amino)caprylic acid constitutes above 90 w/w % of the composition.

5. The pharmaceutical composition according to any of the previous embodiments 2-4, wherein the lubricant is magnesium stearate.

6. The pharmaceutical composition according to any of the previous embodiments, wherein a dose unit comprises
a) 0.5-50 mg of the GLP-1 agonist and/or
b) 50-500 mg of said salt of N-(8-(2-hydroxybenzoyl)amino)caprylic acid.

7. The pharmaceutical composition according to any of the previous embodiments, wherein a dose unit comprises;
a) 0.5-50 mg of the GLP-1 agonist,
b) 50-500 mg of said salt of N-(8-(2-hydroxybenzoyl)amino)caprylic acid and
c) 1-10 mg lubricant, such as magnesium stearate.

8. The pharmaceutical composition according to any of the previous embodiments wherein the GLP-1 agonist is selected from the group consisting of: liraglutide, semaglutide, GLP-1 agonist B and GLP-1 agonist C.

9. The pharmaceutical composition according to any of the previous embodiments, wherein the composition is a solid composition, such as a tablet for oral administration.

10. A pharmaceutical composition comprising
a) a GLP-1 agonist and
b) a salt of N-(8-(2-hydroxybenzoyl)amino)caprylic acid, wherein
   i. the release of the GLP-1 agonist reaches 85 % within 15 minutes and/or
   ii. the dose corrected exposure (AUC) from t=0-30 min is increased relative to a reference composition of type F/H of WO2013/139694 and/or
   iii. the dose corrected exposure at 30 min (AUC) is increased relative to a reference composition of type F/H of WO2013/139694.

11. The pharmaceutical composition according to embodiment 10, wherein the composition has one or more of the features of any of the embodiments 1-9.

12. A pharmaceutical composition according to any of the previous embodiments for use in medicine.

13. A pharmaceutical composition according to any of the previous embodiments for use in a method of treatment of diabetes and/or obesity.

14. A method of treatment of an individual in need thereof, comprising administering a therapeutically active amount of a composition according to any of the previous embodiments to said individual.

### METHODS AND EXAMPLES

### General Methods of Detection and Characterisation

### Assay I: Dissolution Test

A dissolution test is performed in an appropriate dissolution apparatus e.g. USP dissolution apparatus 2, and a standard dissolution test according to the European Pharmacopeia (Ph Eur 2.9.3) may be performed to measure the release of the GLP-1 agonist and SNAC in vitro.

Data described herein is obtained using apparatus 2 in accordance with United States Pharmacopoeia 35 using a paddle rotation speed of 50 rpm. For testing at pH 6.8, the 500 mL dissolution medium of 0.05 M phosphate buffer is used at a temperature of 37±0.5° C. Dissolution media has a content of 0.1% Brij®35. Samples are removed at appropriate intervals. Sample content is determined using a RP-HPLC method for dual detection of SNAC and GLP-1 agonist. The sample content is calculated based on the peak area of the SNAC and GLP-1 agonist peaks in the chromatogram relative to the peak areas of the SNAC and GLP-1 agonist references, respectively. The released amount of SNAC and GLP-1 agonist is calculated as percentages of the actual content in the tablets i.e. 100/200/300 mg/tablet SNAC and 3/5/4 mg/tablet GLP-1 agonist (e.g. analogue A, B or C). The actual content in the tablets is determined using Assay (II). The released amount of GLP-1 agonist is reported as average of 3 tablets.

### Assay II: Analysis of Amount of GLP-1 Agonist and SNAC

Tablets are weighed before extraction of the GLP-1 agonist and SNAC. Tablets are dissolved in a relevant amount of 0.05 M phosphate buffer, pH 7.4, with 20% acetonitrile. Extraction time of two hours is used. Samples are centrifuged and a suitable volume is transferred to HPLC vial. Standards of relevant GLP-1 agonist and SNAC are prepared by using the same diluent as for the samples. HPLC with an UV-detector is used for determining the GLP-1 agonist and SNAC content. The sample content is calculated based on the peak area of the SNAC and GLP-1 agonist peaks in the chromatogram relative to the peak areas of the SNAC and GLP-1 agonist references, respectively.

The content is reported as average of 3 tablets.

### Assay III: Pharmacokinetic Studies in Beagle Dogs

Pharmacokinetic (PK) studies in Beagle dogs are conducted to determine the exposure of the GLP-1 agonists after peroral administration of different dosage forms.

For the pharmacokinetic studies male Beagle dogs are used, 1 to 5 years of age and weighing approximately 10-12 kg at the start of the studies. The dogs are group housed in pens (12 hours light : 12 hours dark), and fed individually and restrictedly once daily with Royal Canin Medium Adult dog (Royal Canin Products, China Branch, or Brogaarden A/S, Denmark). Exercise and group social are permitted daily, whenever possible. The dogs are used for repeated pharmacokinetic studies with a suitable wash-out period between successive dosings. An appropriate acclimatisation period is given prior to initiation of the first pharmacokinetic study. All handling, dosing and blood sampling of the animals are performed by trained and skilled staff. Before the studies the dogs are fasted overnight and from 0 to 4 h after dosing. Besides, the dogs are restricted to water 1 hour before dosing until 4 hours after dosing, but otherwise have ad libitum access to water during the whole period.

The GLP-1 agonist tablets used for the p.o. studies described herein are immediate release SNAC-based tablets dosed orally.

The tablets containing the GLP-1 agonist are administered in the following manner: 10 min prior to tablet administration the dogs are dosed subcutaneously with approximately 3 nmol/kg of SEQ ID NO: 3). The GLP-1 tablets are placed in the back of the mouth of the dog to prevent chewing. The mouth is then closed and 10 mL or 50 mL of tap water is given by a syringe or gavage to facilitate swallowing of the tablet.

### Blood Sampling

Blood is sampled at predefined time points for up till 10 hr post dosing to adequately cover the full plasma concentration-time absorption profile of the GLP-1 agonist. For each blood sampling time point approximately 0.8 mL of whole blood is collected in a 1.5 mL EDTA coated tube, and the tube is gently turned to allowing mixing of the sample with the EDTA. Blood samples (for example 0.8 mL) are collected in EDTA buffer (8mM) and then centrifuged at 4° C. and 2000G for 10 minutes. Plasma is pipetted into Micronic tubes on dry ice, and kept at −20° C. until analysis. Blood samples are taken as appropriate, for example from a venflon in the cephalic vein in the front leg for the first 2 hours and then with syringe from the jugular vein for the rest of the time points (the first few drops are allowed to drain from the venflon to avoid heparin saline from the venflon in the sample).

### General Methods for Tablet Preparation

### Method 1: Dry Granulation

Dry granulation is carried out by roller compaction on a Gerteis MICRO-PACTOR. The roller speed is set at 1 rpm and roller compaction force at 6 kN/cm, fill depth is 8 mm. Subsequent to dry granulation hand sieving of ribbons into granules by using a 0.8 mm wire mesh screen is carried out. Optionally second sieving step on Retch oscillating sieve is applied in order to remove fines, a 90 μm screen is used.

### Method 2: Tablet Preparation

Tablets are produced on a Kilian Style One or a Fette 1021 mounted with a single or 4 sets of punches, resulting in 4 mm round, 7 mm round or 5.75×10 mm, 7.2×12 mm, 7.5×12.5 mm or 9×18 mm oval tablets having no score. Punch size is chosen according to the total tablet weight. For the Kilian Style One, the press speed is set to 10% and for Fette 1021 the press speed is set at 20 rpm. The fill volume is adjusted to obtain tablets having target weights from 28.6 mg to 823.5 mg. Compression forces around 2 to 15 kN are applied to obtain tablets with a crushing strength of around 20-160 N respective to the tablet size.

### EXAMPLES

### Example 1

### Preparation of Compositions

Tablets with different amounts of GLP-1 agonist, SNAC and further excipients were prepared. The content of the prepared compositions is provided in Table 1 (Table 1.1, Table 1.2 and Table 1.3). GLP-1 agonist A is semaglutide, GLP-1 agonist B is Diacylated [Aib8,Arg34,Lys37]GLP-1(7-37) (Example 2 of W02011/080103) and GLP-1 agonist C is Diacylated[Aib8,Glu22,Arg26,Lys27,Glu30,Arg34,Lys36]-GLP-1-(7-37)-peptidyl-Glu-Gly (Example 31 of WO2012/140117). Semaglutide can be prepared according to the method described in WO2006/097537, Example 4. GLP-1 agonists B and C can be prepared as described in WO2011/080103 and WO2012/140117, respectively. SNAC was prepared according to the method described in WO2008/028859.

Reference compositions A, B and C were generally prepared as described in WO2013/139694. The test compositions (A1, A2, B1-134, C1 and C2 were generally prepared as described in method 1 and 2 above, with minor variations in the process prior to roller compaction and tablet preparation as specified below.

Table 1. Tablet compositions expressed as mg per tablet

TABLE 1.1
Overview of compositions with GLP-1 agonist A

| Composition | Reference A | A1 | A2 |
|---|---|---|---|
| GLP-1 agonist A (mg) | 3 | 3 | 3 |
| SNAC (mg) | 300 | 100 | 300 |
| Magnesium stearate (mg) | 9.7 | 2.6 | 7.7 |
| Povidone (mg) | 8 | — | — |
| MCC (mg) | 80 | — | — |

TABLE 1.2
Overview of compositions with GLP-1 agonist B

| Composition | Reference B | B1 | B2 | B3 | B4 |
|---|---|---|---|---|---|
| GLP-1 agonist B (mg) | 5 | 5 | 5 | 5 | 5 |
| SNAC (mg) | 300 | 100 | 200 | 300 | 300 |
| Magnesium stearate (mg) | 9.7 | 2.6 | 5.1 | 7.7 | — |
| Glyceryl dibehenate (mg) | — | — | — | — | 2.2 |
| Povidone (mg) | 8 | — | — | — | — |
| MCC (mg) | 80 | — | — | — | — |

TABLE 1.3
Overview of compositions with GLP-1 agonist C

| Ingredients Composition | Reference C | C1 | C2 |
|---|---|---|---|
| GLP-1 agonist C (mg) | 4 | 4 | 4 |
| SNAC (mg) | 300 | 100 | 300 |
| Magnesium stearate (mg) | 9.7 | 2.6 | 7.7 |
| Povidone (mg) | 8 | — | — |
| MCC (mg) | 80 | — | — |

To prepare test compositions A1, A2, Bl, B3, C1 and C2 the following procedure was followed; magnesium stearate for the granules was passed through a 355 μm or finer sieve. The correct amount of magnesium stearate and SNAC were weighed. Magnesium stearate was manually mixed with SNAC according to the geometric mixing principle. Two cycles of geometric dilution were applied. The remaining quantity of SNAC was transferred to a blending container and mixed with the SNAC and magnesium stearate pre-mix for 30 min at 25 rpm in a Turbula mixer. The blend was roller compacted as described in Method 1.

The correct amount of GLP-1 agonist and SNAC and magnesium stearate granules were weighed. Granules of SNAC and magnesium stearate were added to the blending container and manually mixed with GLP-1 agonist according to the geometric mixing principle. Two cycles of geometric dilution were applied. The remaining quantity of SNAC and magnesium stearate granules was transferred to the blending container. The final mixing of GLP-1 agonist and granules was done for 7 min at 25 rpm or for 20 min at 25 rpm in a Turbula mixer. Tablets were prepared from this composition according to Method 2.

B2 was prepared as follows; the magnesium stearate and SNAC granules were prepared as described for previous test compositions. The correct amount of SNAC and magnesium stearate granules and GLP-1 agonist were weighed. The ⅓ of the granules of SNAC and magnesium stearate were mixed with GLP-1 agonist by addition of the granules to a blending container according to the geometric mixing principle. Mixing for 7 min at 25 rpm in a Turbula mixer was applied. Two cycles of geometric dilution were applied. The remaining quantity of SNAC and magnesium stearate granules was transferred to the blending container and mixed according to geometric mixing principle. The final mixing of GLP-1 agonist and granules was done for 7 min at 25 rpm in a Turbula mixer. Tablets were prepared from this composition according to Method 2.

B4 was prepared as follows; SNAC granules (without magnesium stearate) were prepared as described for previous test compositions and in Method 1. The correct amount of GLP-1 agonist, and SNAC granules were weighed. SNAC granules were added to a blending container and mixed with GLP-1 agonist according to the geometric mixing principle. Two cycles of geometric dilution were applied. The remaining quantity of SNAC granules was added to the blending container. Mechanical mixing of GLP-1 agonist and granules was done for 7 min at 25 rpm in a Turbula mixer. Glyceryl dibehenate (Compritol 888 ATO) was passed through a 350 μm or below sieve. The correct amount of Glyceryl dibehenate was weighed. Glyceryl dibehenate was manually mixed with the mixture of SNAC granules and GLP-1 agonist according to the geometric mixing principle. Three cycles of geometric dilution were applied. The obtained mixture was transferred to a blending container with remaining SNAC granules and GLP-1 agonist mixture. The final mixing with Compritol 888 ATO was done for 2 min at 25 rpm in a Turbula mixer. Tablets were prepared from this composition according to Method 2.

### Example 2

### Dissolution Testing

The objective of the present study was to evaluate the dissolution of the series of the tablet compositions described in Example 1.

Dissolution was measured according to Assay I. Table 2 shows the results for tablets prepared according to Example 1 above, wherein the release is presented as “GLP-1 agonist in solution (%)” describing the amount of GLP-1 agonist in solution after 15, 30 and 60 min relative to the total amount of GLP-1 agonist in the tablet at the start of the experiment. The total amount of GLP-1 agonist and SNAC in the tablets was determined according to Assay

TABLE 2
GLP-1 agonist in solution (%)

| Composition/ | GLP-1 agonist in solution (%) | | |
|---|---|---|---|
| Dosage form | 15 min | 30 min | 60 min |
| A1 | 91.3 | 90.5 | 90.5 |
| A2 | 89.0 | 96.8 | 95.8 |
| B1 | 96.9 | 97.1 | 96.7 |
| B2 | 89.3 | 89.7 | 90.0 |
| B3 | full release | full release | full release |
| B4 | 92.4 | 95.3 | 94.3 |
| C1 | 98.2 | 97.6 | 97.5 |
| C2 | 97.7 | full release | full release |
| Reference A | 35.5 | 58.1 | 75.6 |
| Reference B | 44.0 | 69.2 | 83.5 |
| Reference C | 44.9 | 71.4 | 93.1 |

The results obtained show that the test compositions display a faster release of the GLP-1 agonist compared to what was observed for the reference compositions. A significantly faster release of the GLP-1 agonist is observed for the early time points, i.e. at 15 and 30 minutes.

The difference in release is less significant after 60 minutes. The amount of SNAC in the tablets did not influence the release of the GLP-1 agonist i.e. that tablets comprising 100 mg SNAC dissolve as fast as tablets comprising 300 mg SNAC when measured after 15 minutes or later.

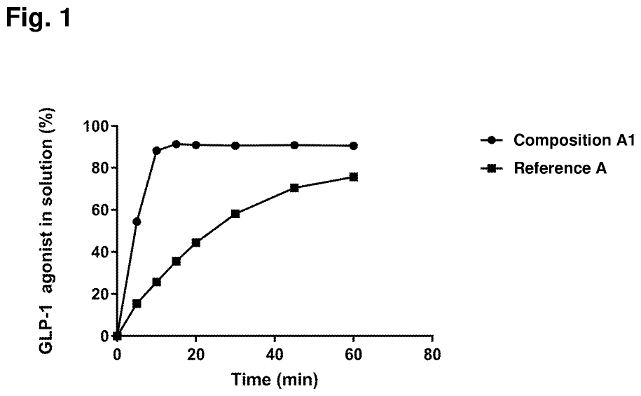

Further data obtained after 5, 10, 15, 20, 30, 45 and 60 minutes for composition A1 and Reference A, is shown in FIG. 1, demonstrating increased release at every time point.

### Example 3

### Oral Exposure

The objective of the present study was to evaluate the oral exposure in beagle dogs of the series of compositions described in Example 1 above using 10 ml water for dosing to the dogs. The number of tests performed for each formulation is indicated by n.

### Analysis and Results

The plasma concentration of the respective GLP-1 agonist was analysed using ELISA or a similar antibody based assay such as LOCI. Individual plasma concentration-time profiles were analysed by a non-compartmental model in WinNonlin v. 5.0 or Phoenix v. 6.2 or 6.3 (Pharsight Inc., Mountain View, Calif., USA), or other relevant software for PK analysis.

The compound exposure measured at t=30 min was determined and normalized by dose/kg bodyweight.

The area under the plasma concentration versus time curve for the first 30 min (AUC, [time×concentration]) was calculated (by the Pharsight programme) after oral administration and normalized by ((dose/kg bodyweight)*100) to obtain the dose corrected exposure. The average values obtained are provided in table 3.1.

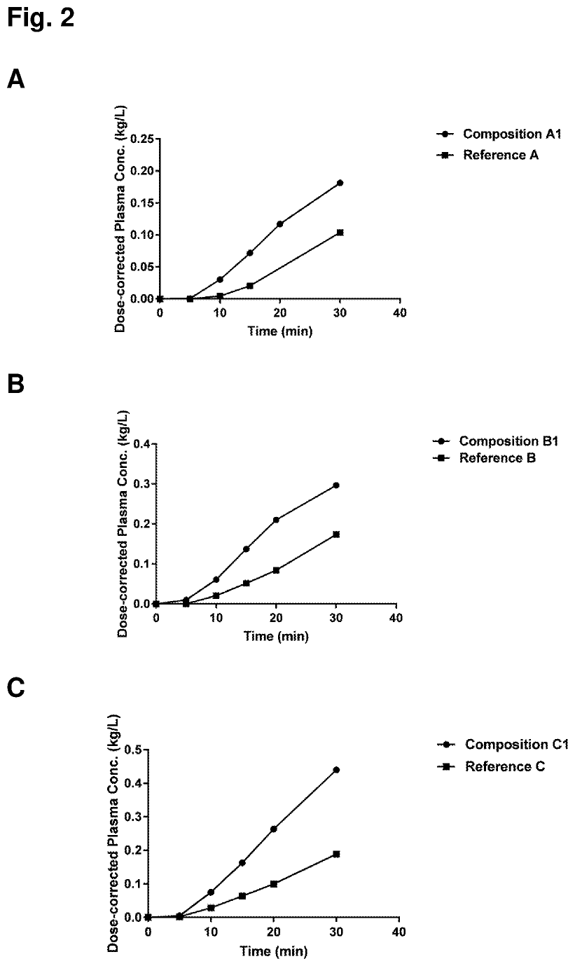

The mean dose-corrected exposure of analogues A, B and C for t=0,5,10,15,20 and 30 min, obtained with the formulations Al ,Reference A, B1 and Reference B, C1 and Reference C are shown in FIG. 2.

Since individual GLP-1 agonists behave differently, i.e. may have a higher or lower general bioavailability, it may be relevant also to compare the relative exposure, to understand the effect of a specific dosage form/dose unit compared to the effect of a different dosage form/dose unit comprising the same GLP-1 agonist. Such results are included in table 3.2, showing that a 2 fold increase in exposure during the first 30 minutes was obtained using the dosage form disclosed herein compared to the reference dosage form for each GLP-1 agonist.

TABLE 3.1
Dose corrected exposure - average values

| | | | Dose corrected | Dose corrected |
|---|---|---|---|---|
| | Formulation/ | SNAC | AUC 0-30 min | exposure t = |
| GLP-1 agonist | dosage form | amount | (arbitrary unit) | 30 min (kg/L) |
| A (n = 24) | Reference A | 300 | 1.68 | 0.104 |
| A (n = 16) | A1 | 100 | 3.84 | 0.181 |
| A (n = 16) | A2 | 300 | 2.63 | 0.153 |
| B (n = 24) | Reference B | 300 | 3.16 | 0.173 |
| B (n = 16) | B1 | 100 | 6.86 | 0.297 |
| B (n = 16) | B3 | 300 | 6.80 | 0.390 |
| B (n = 16) | B4 | 300 | 6.71 | 0.385 |
| C (n = 12) | Reference C | 300 | 3.63 | 0.189 |
| C (n = 12) | C1 | 100 | 9.02 | 0.442 |

TABLE 3.2
Dose corrected exposure - relative exposure

| | | | Fold change of | Fold change of |
|---|---|---|---|---|
| | | | Dose corrected | Dose corrected |
| | | | AUC 0-30 min | exposure t = 30 |
| | | SNAC | normalized to | min normalized |
| GLP-1 agonist | Formulation | amount | reference | to reference |
| A (n = 24) | Reference A | 300 | 1.00 | 1.00 |
| A (n = 16) | A1 | 100 | 2.29 | 1.74 |
| A (n = 16) | A2 | 300 | 1.57 | 1.47 |
| B (n = 24) | Reference B | 300 | 1.00 | 1.00 |
| B (n = 16) | B1 | 100 | 2.17 | 1.72 |
| B (n = 16) | B3 | 300 | 2.15 | 2.25 |
| B (n = 16) | B4 | 300 | 2.12 | 2.22 |
| C (n = 12) | Reference C | 300 | 1.00 | 1.00 |
| C (n = 12) | C1 | 100 | 2.49 | 2.33 |

### Example 4

### Oral Exposure for 25-800 mg SNAC Tablets

Further tablets, with decreasing and increasing amounts of SNAC, were manufactured as described above for A1 and A2.

A5 has the same composition as A2 above. The granules were here manufactured by dry granulation using a MINI-PACTOR® followed by removal of fines using a 90 pm sieve screen. SNAC and magnesium stearate were mixed for 50 min at 25 rpm in a V-shell prior to dry granulation. The SNAC and magnesium stearate granules were mixed with GLP-1 agonist for 10 min at 25 rpm in a V-shell prior to compression of tablets according to Method 2. For composition A7, the GLP-1 agonist A was dry granulated together with SNAC and magnesium stearate using a MINI-PACTOR® followed by removal of fines using a 90 pm sieve screen. SNAC and magnesium stearate were mixed for 50 min at 25 rpm in a V-shell and then GLP-1 was added and mixed for 20 min at 25 rpm prior to dry granulation. The granules were subsequently compressed into tablets according to Method 2. For both compositions A5 and A7, the MINI-PACTOR was operated at a compaction force of 6 kN/cm, a gap of 1 mm, a roll speed of 3 rpm and with a 0.63 μm wire mesh screen.

TABLE 4.1
Tablet compositions expressed as mg per tablet

| | Reference | | | | | |
|---|---|---|---|---|---|---|
| Composition | A | A3 | A4 | A5 | A6 | A7 |
| GLP-1 agonist A (mg) | 3 | 3 | 3 | 3 | 3 | 3 |
| SNAC (mg) | 300 | 25 | 100 | 300 | 800 | 300 |
| Magnesium stearate (mg) | 9.7 | 0.6 | 2.6 | 7.7 | 20.5 | 7.7 |
| Povidone (mg) | 8 | — | — | — | — | — |
| MCC (mg) | 80 | — | — | — | — | — |

The objective of the present study was to evaluate the oral exposure in beagle dogs of the series of compositions described in Table 4. The oral exposure was evaluated in beagle dogs as described in Example 1 above using 50 ml water for dosing to the dogs. The number of tests performed for each formulation is indicated by n.

Results was analysed as described in example 3 and the average values obtained are provided in table 4.2 and the relative values are provided in table 4.3 below.

TABLE 4.2
Dose corrected exposure - average values

| | | | Dose corrected | Dose corrected |
|---|---|---|---|---|
| | Formulation/ | SNAC | AUC 0-30 min | exposure t = |
| GLP-1 agonist | dosage form | amount | (arbitrary units) | 30 min (kg/L) |
| A (n = 19) | Reference A | 300 | 0.82 | 0.048 |
| A (n = 16) | A3 | 25 | 4.30 | 0.168 |
| A (n = 34) | A4 | 100 | 3.30 | 0.151 |
| A (n = 16) | A5 | 300 | 3.27 | 0.173 |
| A (n = 16) | A6 | 800 | 2.48 | 0.147 |
| A(n = 16) | A7 | 300 | 2.60 | 0.141 |

TABLE 4.3
Dose corrected exposure - relative exposure

| | | | Fold change of | Fold change of |
|---|---|---|---|---|
| | | | Dose corrected | Dose corrected |
| | | | AUC 0-30 min | exposure t = 30 |
| | Formulation/ | SNAC | normalized to | min normalized |
| GLP-1 agonist | dosage form | amount | reference | to reference |
| A (n = 19) | Reference A | 300 | 1.00 | 1.00 |
| A (n = 16) | A3 | 25 | 5.24 | 3.50 |
| A (n = 34) | A4 | 100 | 4.02 | 3.15 |
| A (n = 16) | A5 | 300 | 3.99 | 3.60 |
| A (n = 16) | A6 | 800 | 3.02 | 3.06 |
| A (n = 16) | A7 | 300 | 3.17 | 2.94 |

The data confirmed that an increase in exposure during the first 30 minutes was obtained using the dosage form disclosed herein compared to the reference dosage form.

1. A pharmaceutical composition comprising
a) 0.5-50 mg of a GLP-1 agonist selected from the group consisting of: 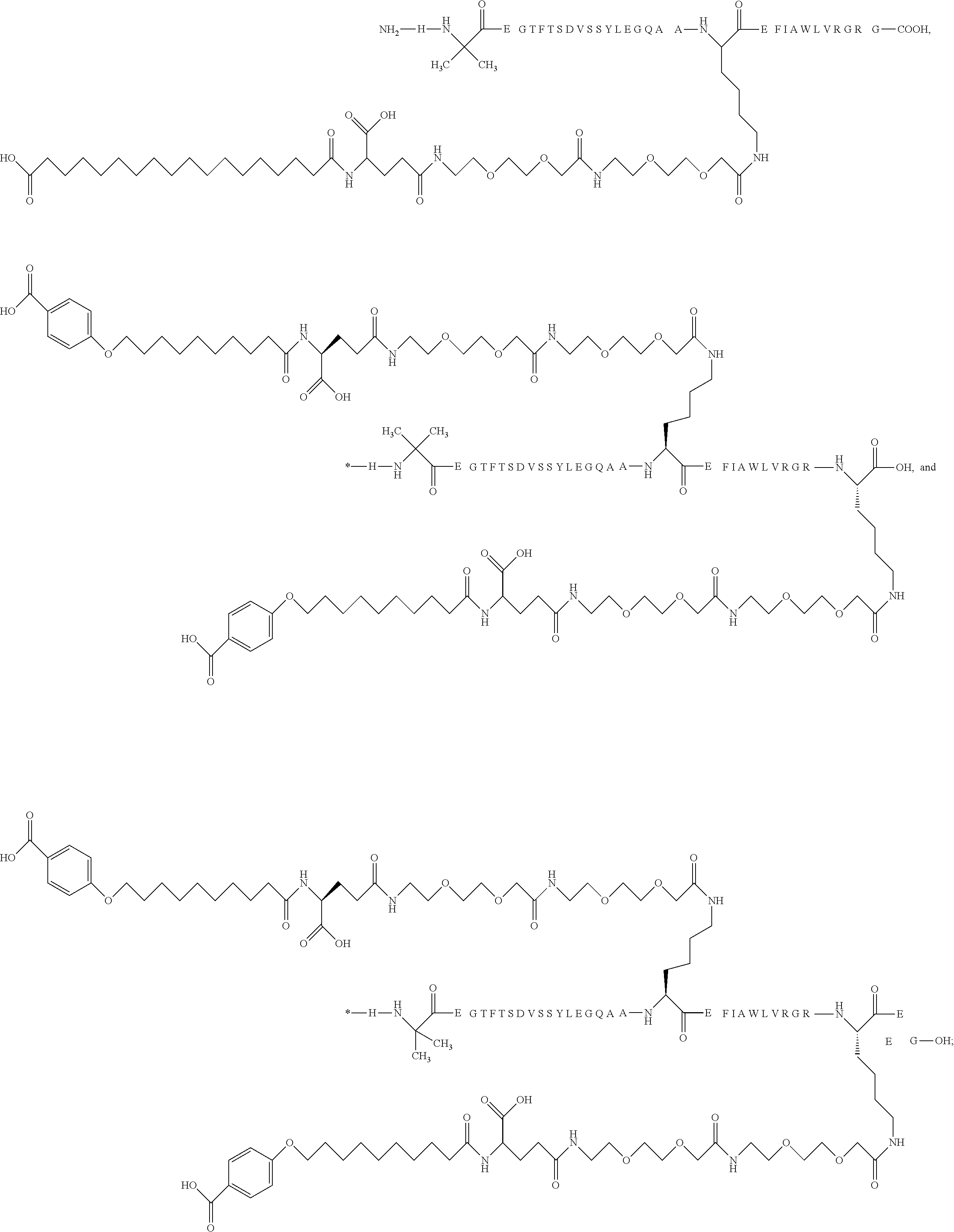
b) 20-800 mg of a salt of N-(8-(2-hydroxybenzoyl)amino)caprylic acid; and
c) 0.6-20 mg of lubricant;
wherein the salt of N-(8-(2-hydroxybenzoyl)amino)caprylic acid constitutes at least 90 w/w % of the excipients of the pharmaceutical composition.

2. The pharmaceutical composition according to claim 1, wherein the GLP-1 agonist is

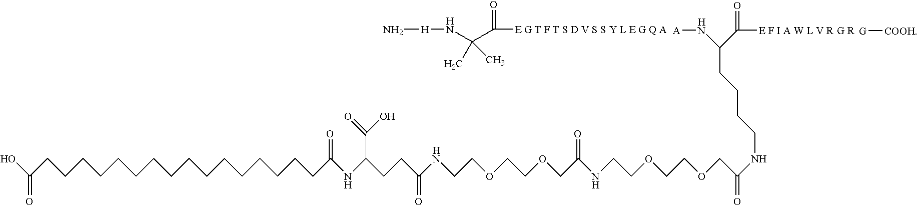

3. The pharmaceutical composition according to claim 1, wherein the GLP-1 agonist is

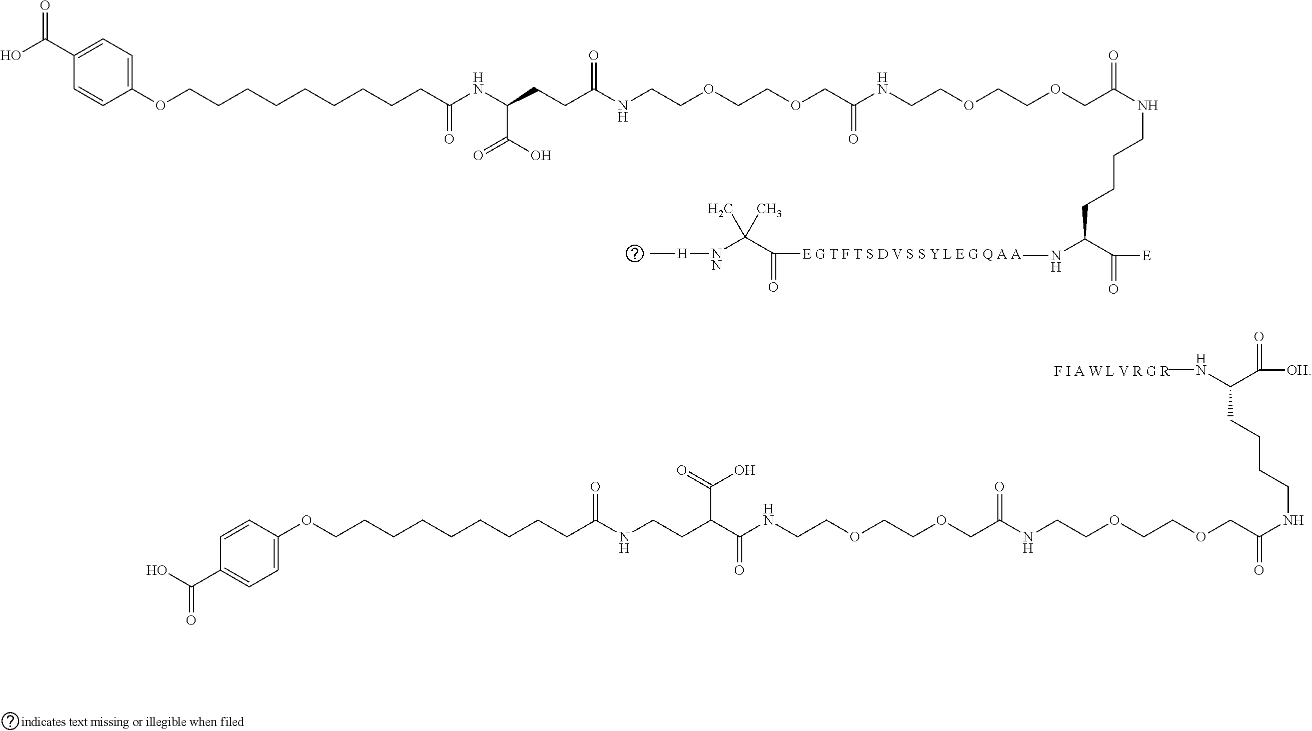

4. The pharmaceutical composition according to claim 1, wherein the GLP-1 agonist is

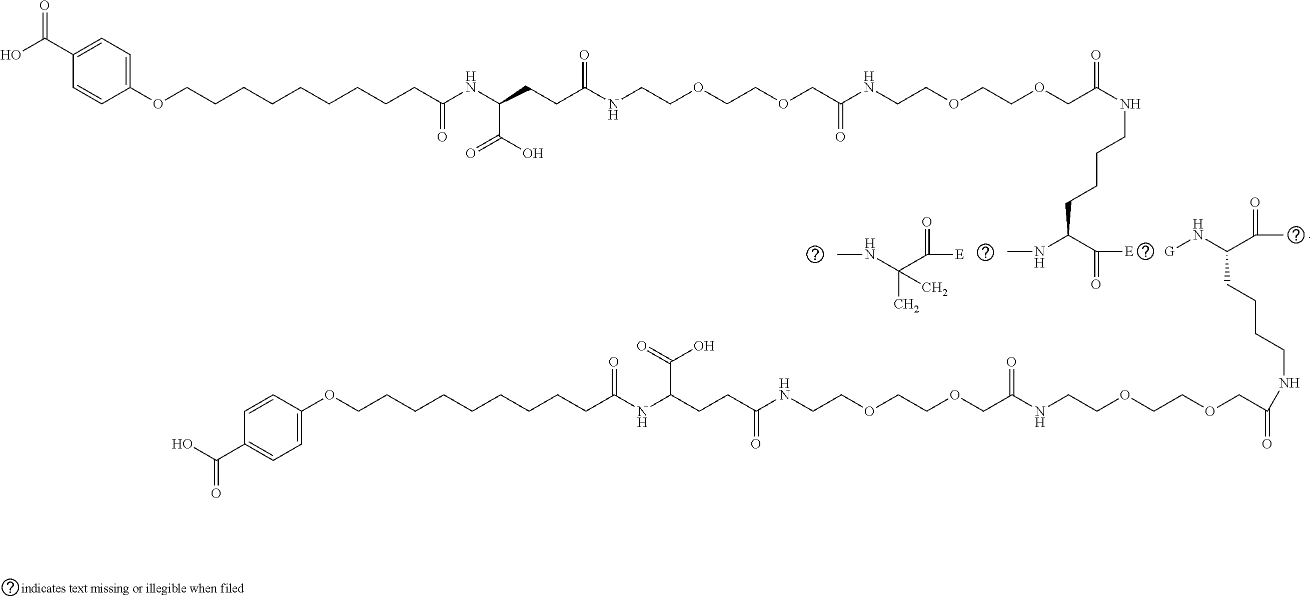

5. The pharmaceutical composition according to claim 1, wherein the lubricant is magnesium stearate or glyceryl dibehenate.

6. The pharmaceutical composition according to claim 1, wherein the salt of N-(8-(2-hydroxybenzoyl)amino)caprylic acid constitutes at least 95 w/w % of the composition.

7. The pharmaceutical composition according to claim 1, wherein the salt of N-(8-(2-hydroxybenzoyl)amino)caprylic acid is sodium N-(8-(2-hydroxybenzoyl)amino)caprylate.

8. The pharmaceutical composition according to claim 1, wherein the lubricant is magnesium stearate, and wherein the pharmaceutical composition comprises 1-8 mg magnesium stearate per 100 mg of the salt of N-(8-(2-hydroxybenzoyl)amino)caprylic acid.

9. The pharmaceutical composition according to claim 7, wherein the pharmaceutical composition comprises 2-5 mg magnesium stearate per 100 mg of the salt of N-(8-(2-hydroxybenzoyl)amino)caprylic acid.

10. The pharmaceutical composition according to claim 9, wherein the salt of N-(8-(2-hydroxybenzoyl)amino)caprylic acid is sodium N-(8-(2-hydroxybenzoyl)amino)caprylate.

11. A pharmaceutical composition consisting essentially of
a) 0.5-50 mg of a GLP-1 agonist selected from the group consisting of: 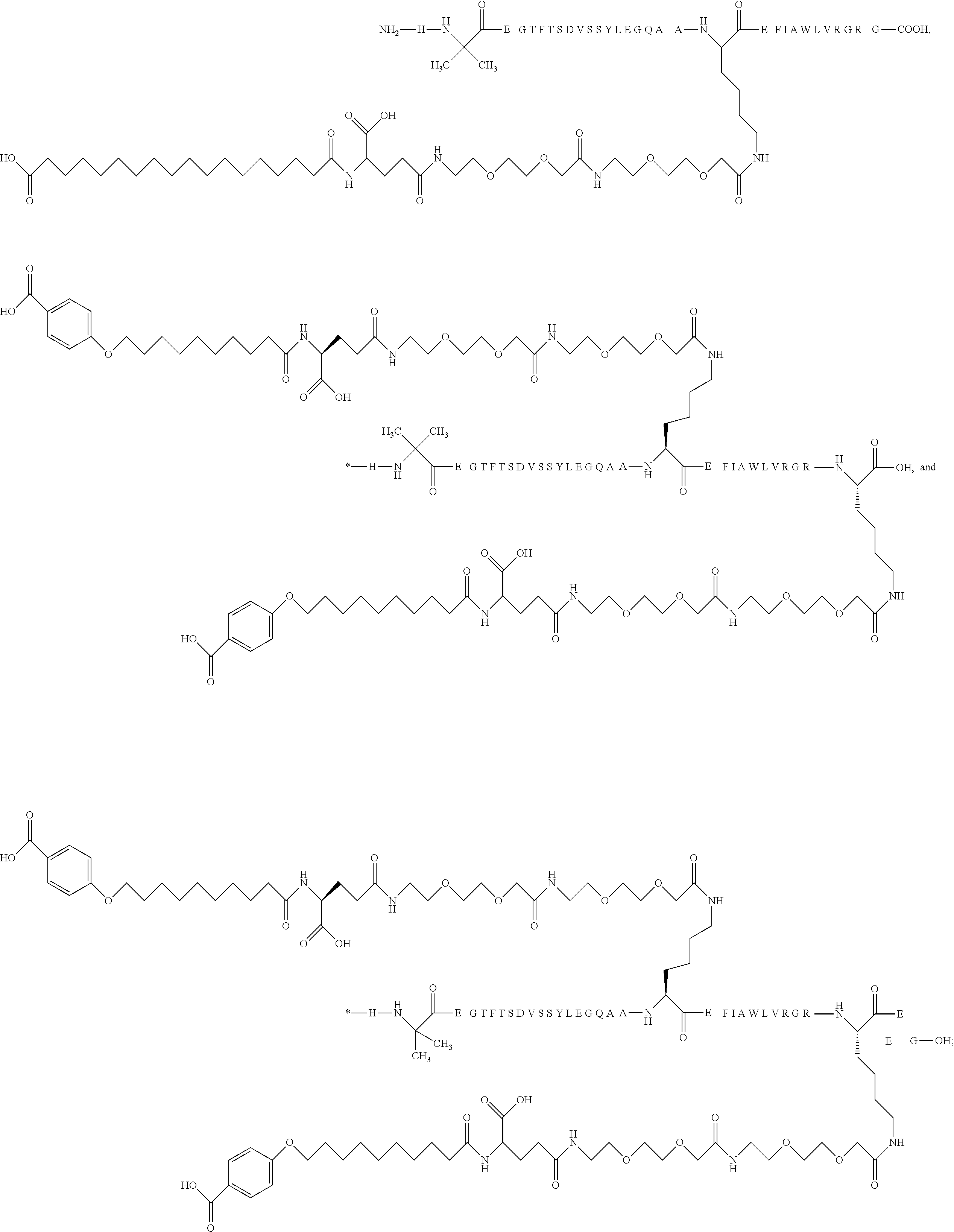
b) 20-800 mg of a salt of N-(8-(2-hydroxybenzoyl)amino)caprylic acid; and
c) 0.6-20 mg of lubricant.

12. The pharmaceutical composition according to claim 11, wherein the salt of N-(8-(2-hydroxybenzoyl)amino)caprylic acid is sodium N-(8-(2-hydroxybenzoyl)amino)caprylate.

13. The pharmaceutical composition according to claim 11, wherein the lubricant is magnesium stearate or glyceryl dibehenate.

14. The pharmaceutical composition according to claim 11, wherein the composition consists essentially of:
a) 1-10 mg of the GLP-1 agonist;
b) 50-400 mg of the salt of N-(8-(2-hydroxybenzoyl)amino)caprylic acid; and
c) 1-10 mg of the lubricant;
wherein the salt of N-(8-(2-hydroxybenzoyl)amino)caprylic acid is sodium N-(8-(2-hydroxybenzoyl)amino)caprylate; and
wherein the lubricant is magnesium stearate.

15. The pharmaceutical composition according to claim 14, wherein the sodium N-(8-(2-hydroxybenzoyl)amino)caprylate is in amount selected from the group consisting of 100 mg, 200 mg, and 300 mg.

16. The pharmaceutical composition according to claim 14, wherein the GLP-1 agonist is 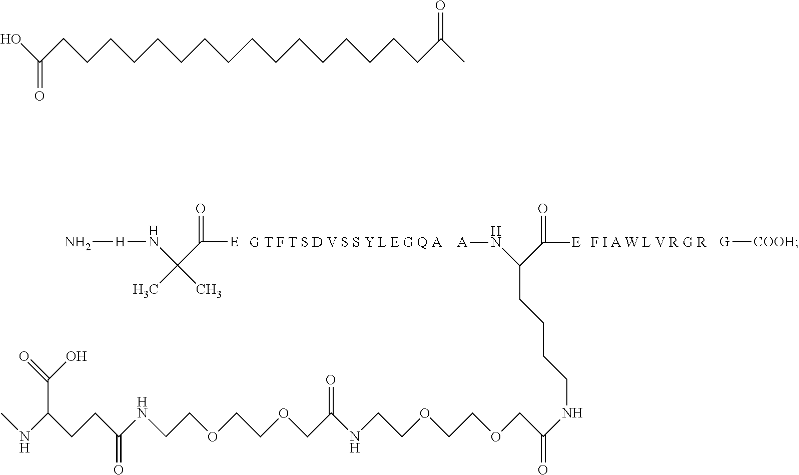
and
wherein the pharmaceutical composition comprises 1-8 mg magnesium stearate per 100 mg of the sodium N-(8-(2-hydroxybenzoyl)amino)caprylate.

17. The pharmaceutical composition according to claim 16, wherein the pharmaceutical composition comprises 2-5 mg magnesium stearate per 100 mg of the sodium N-(8-(2-hydroxybenzoyl)amino)caprylate.

18. The pharmaceutical composition according to claim 14,
wherein the GLP-1 agonist is 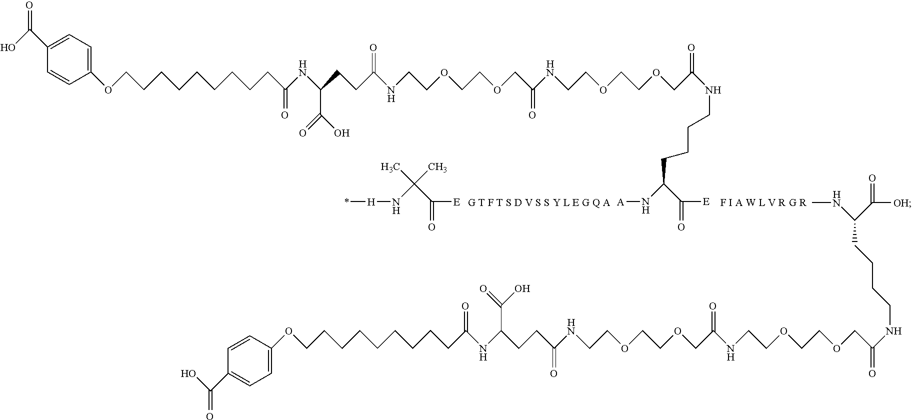
and
wherein the pharmaceutical composition comprises 1-8 mg magnesium stearate per 100 mg of the sodium N-(8-(2-hydroxybenzoyl)amino)caprylate.

19. The pharmaceutical composition according to claim 18, wherein the pharmaceutical composition comprises 2-5 mg magnesium stearate per 100 mg of the sodium N-(8-(2-hydroxybenzoyl)amino)caprylate.

20. The pharmaceutical composition according to claim 14,
wherein the GLP-1 agonist is 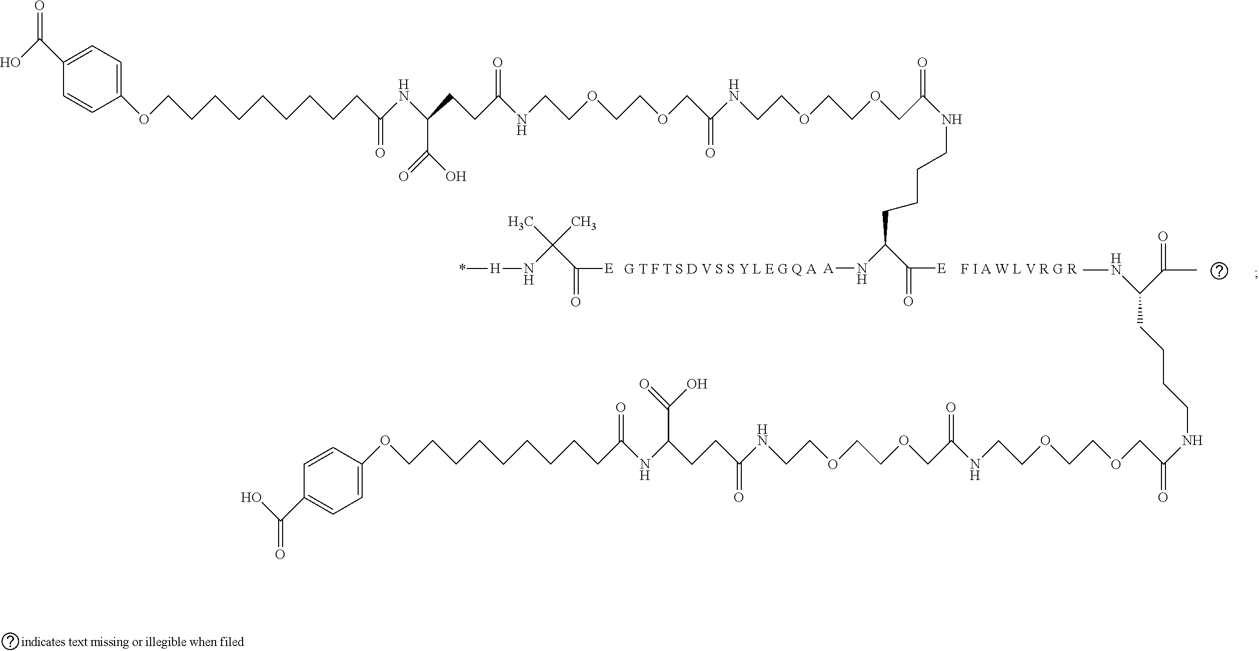
and
wherein the pharmaceutical composition comprises 1-8 mg magnesium stearate per 100 mg of the sodium N-(8-(2-hydroxybenzoyl)amino)caprylate.

21. The pharmaceutical composition according to claim 20, wherein the pharmaceutical composition comprises 2-5 mg magnesium stearate per 100 mg of the sodium N-(8-(2-hydroxybenzoyl)amino)caprylate.

22. A method of treating diabetes, comprising administering to a subject in need thereof a therapeutically active amount of the pharmaceutical composition according to claim 14.

23. The method according to claim 22, wherein the composition comprises an amount of the sodium N-(8-(2-hydroxybenzoyl)amino)caprylate selected from the group consisting of 100 mg, 200 mg, and 300 mg.

24. The method according to claim 23, wherein the pharmaceutical composition comprises 1-8 mg magnesium stearate per 100 mg of the sodium N-(8-(2-hydroxybenzoyl)amino)caprylate.

25. The method according to claim 24, wherein the pharmaceutical composition comprises 2-5 mg magnesium stearate per 100 mg of the sodium N-(8-(2-hydroxybenzoyl)amino)caprylate.

26. A method of treating obesity, comprising administering to a subject in need thereof a therapeutically active amount of a pharmaceutical composition according to claim 14.

27. The method according to claim 26, wherein the pharmaceutical composition comprises 1-8 mg magnesium stearate per 100 mg of the sodium N-(8-(2-hydroxybenzoyl)amino)caprylate.

28. The method according to claim 27, wherein the pharmaceutical composition comprises 2-5 mg magnesium stearate per 100 mg of the sodium N-(8-(2-hydroxybenzoyl)amino)caprylate.
